SEQUENCE LISTING (34 sequences)

SEQ ID NO: 1 - protein, 22 aa, Homo sapiens
MDMRVPAQLLGLLLLWLSGARC

SEQ ID NO: 2 - protein, 23 aa, synthetic construct
  REGION 1..23: note = Synthetic
MDMRVPAQLLGLLLLWLSGARCQ

SEQ ID NO: 3 - protein, 27 aa, Homo sapiens
MATGSRTSLLLAFGLLCLPWLQEGSQA

SEQ ID NO: 4 - protein, 18 aa, synthetic construct
  REGION 1..18: note = Synthetic
GGSSRSSSSGGGGSGGGG

SEQ ID NO: 5 - protein, 20 aa, synthetic construct
  REGION 1..20: note = Synthetic
GGGGSGGGGSGGGGSGGGGS

SEQ ID NO: 6 - protein, 10 aa, synthetic construct
  REGION 1..10: note = Synthetic
EQKLISEEDL

SEQ ID NO: 7 - protein, 62 aa, Homo sapiens
ALSNSIMYFSHFVPVFLPAKPTTTPAPRPPTPAPTIASQPLSLRPEASRPAAGGAVHTRGLD

SEQ ID NO: 8 - protein, 134 aa, Mus musculus
LCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 9 - protein, 134 aa, synthetic construct
  REGION 1..134: note = Synthetic
LCYLLDGILFIYGVIITALYLRAKFSRSAETAANLQDPNQLFNELNLGRREEFDVLEKKRARDPEMGGKQQRRRNPQEGVFNALQKDKMAEAFSEIGTKGERRRGKGHDGLFQGLSTATKDTFDALHMQTLAPR

SEQ ID NO: 10 - protein, 114 aa, Homo sapiens
DVVMTQLPLSLPVILGDQASISCRSSQSLIHSNGNTYLHWYLQKPGQSPKLLIYKVSNRFSGVPDRFSGSGSGTDFTLKISRVEAEDLGVYFCSQSTHVPFTFGSGTKLEIKRA

SEQ ID NO: 11 - protein, 119 aa, Homo sapiens
QVQLQQSGPELVKPGASVKLSCKASGYTFTSYDINWVKQRPGQGLEWIGWIYPRDGSIKFNEKFKGKATLTVDTSSSTAYMELHSLTSEDSAAYFCARRGRLLLYGFAYWGQGTLVTVS

SEQ ID NO: 12 - protein, 114 aa, Homo sapiens
DVVMTQTPLSLPVSLGDQASISCRSSQSLVHSNGNTYLHWYLQKPGQSPNLLIYKVSNRFSGVPDRFSGSGSGTDFTLKISRVEAEDLGVYFCSQGTHVPYTFGGGTKLEIKRA

SEQ ID NO: 13 - protein, 117 aa, Homo sapiens
QVQLQQSGAELVRPGASVTLSCKASGYTFTDYEMHWVKQTPVHGLEWIGTIDPETAGTAYNNQKFKGKAILTAGKSSSTAYMELRSLTSEDSAVYYCTGVTTWFAYWGQGTLVTVSA

SEQ ID NO: 14 - protein, 108 aa, Homo sapiens
QIVLTQSPAIMSASPGEKVTMTCSASSRVTYMHWYQQRSGTSPKRWIYDTSKLASGVPARFSGSGSGTSYSLTISSMEAEDAATYYCQQWGNNPQYTFGGGTRLEIKR

SEQ ID NO: 15 - protein, 120 aa, Homo sapiens
QVTLKESGPGILQPSQTLSLTCSFSGFSLSTSGTGVGWIRQPSGKGLEWLSHIWWDDVKRYNPALKSRLTISRDTSYSQVFLRIASVDTADTATYYCARILDGTGPMDYWGQGTSVTVSS

SEQ ID NO: 16 - protein, 217 aa, Homo sapiens
EGSADYKDHDGDYKDHDIDYKDDDDKFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 17 - protein, 460 aa, synthetic construct
  REGION 1..460: note = Synthetic
  VAR_SEQ 253..254: note = Xaa can be any naturally occurring amino acid
DVVMTQLPLSLPVILGDQASISCRSSQSLIHSNGNTYLHWYLQKPGQSPKLLIYKVSNRFSGVPDRFSGSGSGTDFTLKISRVEAEDLGVYFCSQSTHVPFTFGSGTKLEIKRAGGSSRSSSSGGGGSGGGGQVQLQQSGPELVKPGASVKLSCKASGYTFTSYDINWVKQRPGQGLEWIGWIYPRDGSIKFNEKFKGKATLTVDTSSSTAYMELHSLTSEDSAAYFCARRGRLLLYGFAYWGQGTLVTVSAXXEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 18 - protein, 460 aa, synthetic construct
  REGION 1..460: note = Synthetic
  VARIANT 253..254: note = Xaa can be any naturally occurring amino acid
DVVMTQLPLSLPVILGDQASISCRSSQSLIHSNGNTYLHWYLQKPGQSPKLLIYKVSNRFSGVPDRFSGSGSGTDFTLKISRVEAEDLGVYFCSQSTHVPFTFGSGTKLEIKRAGGSSRSSSSGGGGSGGGGQVQLQQSGPELVKPGASVKLSCKASGYTFTSYDINWVKQRPGQGLEWIGWIYPRDGSIKFNEKFKGKATLTVDTSSSTAYMELHSLTSEDSAAYFCARRGRLLLYGFAYWGQGTLVTVSAXXEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLFNELNLGRREEFDVLEKKRARDPEMGGKQQRRRNPQEGVFNALQKDKMAEAFSEIGTKGERRRGKGHDGLFQGLSTATKDTFDALHMQTLAPR

SEQ ID NO: 19 - protein, 704 aa, synthetic construct
  REGION 1..704: note = Synthetic
  VARIANT 250..251: note = Xaa can be any naturally occurring amino acid
DVVMTQTPLSLPVSLGDQASISCRSSQSLVHSNGNTYLHWYLQKPGQSPNLLIYKVSNRFSGVPDRFSGSGSGTDFTLKISRVEAEDLGVYFCSQGTHVPYTFGGGTKLEIKRAGGSSRSSSSGGGGSGGGGQVQLQQSGAELVRPGASVTLSCKASGYTFTDYEMHWVKQTPVHGLEWIGTIDPETAGTAYNNQKFKGKAILTAGKSSSTAYMELRSLTSEDSAVYYCTGVTTWFAYWGQGTLVTVSAXXALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPREGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 20 - protein, 704 aa, synthetic construct
  REGION 1..704: note = Synthetic
  VARIANT 250..251: note = Xaa can be any naturally occurring amino acid
DVVMTQTPLSLPVSLGDQASISCRSSQSLVHSNGNTYLHWYLQKPGQSPNLLIYKVSNRFSGVPDRFSGSGSGTDFTLKISRVEAEDLGVYFCSQGTHVPYTFGGGTKLEIKRAGGSSRSSSSGGGGSGGGGQVQLQQSGAELVRPGASVTLSCKASGYTFTDYEMHWVKQTPVHGLEWIGTIDPETAGTAYNNQKFKGKAILTAGKSSSTAYMELRSLTSEDSAVYYCTGVTTWFAYWGQGTLVTVSAXXALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLFNELNLGRREEFDVLEKKRARDPEMGGKQQRRRNPQEGVFNALQKDKMAEAFSEIGTKGERRRGKGHDGLFQGLSTATKDTFDALHMQTLAPREGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 21 - protein, 455 aa, synthetic construct
  REGION 1..455: note = Synthetic
  VARIANT 248..249: note = Xaa can be any naturally occurring amino acid
IVLTQSPAIMSASPGEKVTMTCSASSRVTYMHWYQQRSGTSPKRWIYDTSKLASGVPARFSGSGSGTSYSLTISSMEAEDAATYYCQQWGNNPQYTFGGGTRLEIKRGGGGSGGGGSGGGGSGGGGSQVTLKESGPGILQPSQTLSLTCSFSGFSLSTSGTGVGWIRQPSGKGLEWLSHIWWDDVKRYNPALKSRLTISRDTSYSQVFLRIASVDTADTATYYCARILDGTGPMDYWGQGTSVTVSSXXEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 22 - protein, 455 aa, synthetic construct
  REGION 1..455: note = Synthetic
  VARIANT 248..249: note = Xaa can be any naturally occurring amino acid
IVLTQSPAIMSASPGEKVTMTCSASSRVTYMHWYQQRSGTSPKRWIYDTSKLASGVPARFSGSGSGTSYSLTISSMEAEDAATYYCQQWGNNPQYTFGGGTRLEIKRGGGGSGGGGSGGGGSGGGGSQVTLKESGPGILQPSQTLSLTCSFSGFSLSTSGTGVGWIRQPSGKGLEWLSHIWWDDVKRYNPALKSRLTISRDTSYSQVFLRIASVDTADTATYYCARILDGTGPMDYWGQGTSVTVSSXXEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLFNELNLGRREEFDVLEKKRARDPEMGGKQQRRRNPQEGVFNALQKDKMAEAFSEIGTKGERRRGKGHDGLFQGLSTATKDTFDALHMQTLAPR

SEQ ID NO: 23 - protein, 425 aa, synthetic construct
  REGION 1..425: note = Synthetic
  VARIANT 218..219: note = Xaa can be any naturally occurring amino acid
EGSADYKDHDGDYKDHDIDYKDDDDKFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFXXEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 24 - protein, 425 aa, synthetic construct
  REGION 1..425: note = Synthetic
  VARIANT 218..219: note = Xaa can be any naturally occurring amino acid
EGSADYKDHDGDYKDHDIDYKDDDDKFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFXXEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLFNELNLGRREEFDVLEKKRARDPEMGGKQQRRRNPQEGVFNALQKDKMAEAFSEIGTKGERRRGKGHDGLFQGLSTATKDTFDALHMQTLAPR

SEQ ID NO: 25 - protein, 218 aa, Homo sapiens
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 447 aa, Homo sapiens
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 891 aa, synthetic construct
  REGION 1..891: note = Synthetic
  REGION 684..685: note = misc_feature - Xaa can be any naturally occurring amino acid
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGSSRSSSSGGGGSGGGGEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECXXEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 28 - protein, 891 aa, synthetic construct
  REGION 1..891: note = Synthetic
  REGION 684..685: note = misc_feature - Xaa can be any naturally occurring amino acid
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGSSRSSSSGGGGSGGGGEIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECXXEQKLISEEDLALSNSIMYFSHFVPVFLPAKPTTTPAPRPPTPAPTIASQPLSLRPEASRPAAGGAVHTRGLDLCYLLDGILFIYGVIITALYLRAKFSRSAETAANLQDPNQLFNELNLGRREEFDVLEKKRARDPEMGGKQQRRRNPQEGVFNALQKDKMAEAFSEIGTKGERRRGKGHDGLFQGLSTATKDTFDALHMQTLAPR

SEQ ID NO: 29 - protein, 21 aa, Mus musculus
LCYLLDGILFIYGVIITALYL

SEQ ID NO: 30 - protein, 113 aa, Mus musculus
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 31 - protein, 113 aa, synthetic construct
  REGION 1..113: note = Synthetic
RAKFSRSAETAANLQDPNQLFNELNLGRREEFDVLEKKRARDPEMGGKQQRRRNPQEGVFNALQKDKMAEAFSEIGTKGERRRGKGHDGLFQGLSTATKDTFDALHMQTLAPR

SEQ ID NO: 32 - protein, 39 aa, Homo sapiens
KCFCKHRSCFRRNEASRETNNSLTFGPEELALAEQTVFL

SEQ ID NO: 33 - protein, 239 aa, synthetic construct
  REGION 1..239: note = Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 34 - protein, 22 aa, Homo sapiens
MATGSRTSLLLAFGLLCLPWLQ

(19) United States
(12) Patent Application Publication
(10) Pub. No.: US 2025/0043000 A1
(43) Pub. Date: Feb. 6, 2025

(54) NON-CYTOTOXIC MODIFIED CELLS AND USE THEREOF

(71) Applicant: THE NATIONAL INSTITUTE FOR BIOTECHNOLOGY IN THE NEGEV LTD., Beer Sheva (IL)

(72) Inventors: Alon MONSONEGO, Nir Banim (IL); Angel PORGADOR, Lehavim (IL); Roee ATLAS, Givataim (IL)

(73) Assignee: THE NATIONAL INSTITUTE FOR BIOTECHNOLOGY IN THE NEGEV LTD., Beer Sheva (IL)

(21) Appl. No.: 18/810,629

(22) Filed: Aug. 21, 2024

Related U.S. Application Data
(63) Continuation of application No. 16/340,196, filed on Apr. 8, 2019, now Pat. No. 12,098,202.
(60) Provisional application No. 62/406,005, filed on Oct. 10, 2016.

(51) Int. Cl. C07K 16/28; A61K 35/15; A61K 35/17; A61K 39/00; A61K 48/00; A61P 3/00; A61P 9/00; A61P 25/28; A61P 35/00; C07K 14/705; C07K 14/725; C07K 16/18; C12N 5/0783

(52) U.S. Cl. CPC C07K 16/2809; A61K 35/15; A61K 35/17; A61K 39/4611; A61K 39/4631; A61K 39/464402; A61K 48/00; A61K 2239/22; A61P 3/00; A61P 9/00; A61P 25/28; A61P 35/00; C07K 14/7051; C07K 14/70532; C07K 16/18; C07K 16/2818; C12N 5/0636; C12N 5/0638; C12N 5/0646; C07K 2317/74; C07K 2317/75; C07K 2319/03; C07K 2319/41; C07K 2319/60; C12N 2501/25; C12N 2501/335; C12N 2501/515; C12N 2502/30; C12N 2510/00

(57) ABSTRACT

Modified cells comprising a transmembrane polypeptide comprising at least one extracellular target receptor-binding domain, a transmembrane domain and an intracellular domain, wherein said intracellular domain is not capable of transducing any signal are provided. Methods of inducing or inhibiting signaling by a target receptor in a target cell comprising contacting the target cell with a modified cell of the invention are also provided.

### CROSS-REFERENCE TO RELATED APPLICATION

This application is a continuation of U.S. patent application Ser. No. 16/340,196, filed on Apr. 8, 2019, which is a National Phase of PCT Patent Application No. PCT/IL2017/051133 having International filing date of Oct. 10, 2017, which claims the benefit of priority from U.S. Provisional Patent Application No. 62/406,005 filed on Oct. 10, 2016, the contents of the above applications are all incorporated by reference as if fully set forth herein in their entirety.

### REFERENCE TO AN ELECTRONIC SEQUENCE LISTING

The contents of the electronic sequence listing (NIBN-P-017-US1 Corrected 2.xml; Size: 48,718 bytes; and Date of Creation: Oct. 15, 2024) is herein incorporated by reference in its entirety.

### FIELD OF INVENTION

The present invention is directed to the field of cell therapy.

### BACKGROUND OF THE INVENTION

There are many known antibodies, antibody fragments and ligands that can specifically bind various receptors and act as either agonists or antagonists. Such agonistic/antagonistic have been used for the treatment of various diseases. However, a major limitation for the use of such molecules is the complexity of targeting the molecule to a specific organ, tissue and/or specific cell type. It is difficult to transfer large molecules across the blood brain barrier, and to deep parenchymal areas as well. Further these molecules often have short half-life, high immunogenicity or never reach the intended target region at all.

Cellular delivery of these potential therapeutic molecules is one possible avenue that may circumvent many of these problems. However, such a solution relies on the ability of the cell to home to the desired treatment area or area of disease. Further, in order to perform an autologous or allogenic treatment that does not induce an immune response, it must be possible to culture and expand the primary cells outside of the body of the subject. Immune cells offer an unexplored avenue for the delivery of these therapeutic molecules, because current delivery methods would result in activation of the immune cell and thus localized inflammation at best and direct killing of the cell that is being treated at worst. A method of delivering therapeutic agonists and antagonists to diseased cells anywhere in the body, without harming those cells or increasing inflammation in the diseased area is greatly desired.

### SUMMARY OF THE INVENTION

The present invention provides methods of modulating signaling by a target receptor in a target cell and methods of treating a subject suffering from a disease or disorder wherein the disease or disorder is associated with the target receptor.

According to a first aspect, there is provided a method of modulating signaling by a target receptor in a target cell, the method comprising contacting the target cell with a modified cell comprising a chimeric transmembrane polypeptide comprising at least one extracellular target receptor-binding domain, a transmembrane domain and an intracellular domain, wherein the intracellular domain does not transduce a signal, and wherein the modified cell serves as a ligand and thereby modulates signaling by a target receptor in a target cell.

According to another aspect, there is provided a method of treating a subject suffering from a disease or disorder, the method comprising:
a. providing a cell capable of homing to the site of the disease or disorder;
b. expressing in the cell a chimeric transmembrane polypeptide comprising at least one extracellular target receptor-binding domain, a transmembrane domain and an intracellular domain, wherein the intracellular domain is not capable of transducing any signal and wherein the target receptor is associated with the disease or disorder; and
c. administering the cell expressing the chimeric transmembrane polypeptide to the subject;
   thereby treating the subject suffering from a disease or disorder.

In some embodiments, the signaling by a target cell comprises a signaling cascade in the target cell. In some embodiments, the modulating comprises inducing or inhibiting. In some embodiments, inducing signaling comprises phosphorylation of a residue within a signaling domain of the target receptor. In some embodiments, inducing signaling comprises upregulation of a level of a downstream target of the target receptor. In some embodiments, inhibiting signaling comprises down-regulation of a level of a downstream target of said target receptor.

In some embodiments, the chimeric transmembrane polypeptide comprises an extracellular domain and intracellular domain that are from different proteins.

In some embodiments, the extracellular domain comprises an agonist or antagonist of said target receptor.

In some embodiments, the target receptor-binding domain comprises an immunoglobulin variable heavy chain domain (VH) and an immunoglobulin variable light chain domain (VL). In some embodiments, the VH and VL are connected by a peptide linker. In some embodiments, the linker comprises the amino acid sequence GGSSRSSSSGGGGSGGGG (SEQ ID NO: 4) or GGGGGGGGSGGGGSGGGGS (SEQ ID NO: 5).

In some embodiments, the transmembrane domain is a single-pass transmembrane domain. In some embodiments, the transmembrane domain comprises a CD3 transmembrane domain. In some embodiments, the CD3 transmembrane domain comprises the sequence LCYLLDGILFIYGVIITALYL (SEQ ID NO: 29).

In some embodiments, the chimeric transmembrane polypeptide further comprises an extracellular and membrane proximal hinge region. In some embodiments, the hinge region comprises a CD-8 hinge region. In some embodiments, the CD-8 hinge region comprises the amino acid sequence

(SEQ ID NO: 7)
ALSNSIMYFSHFVPVFLPAKPTTTPAPRPPTPAPTIASQPLSLR
PEASRPAAGGAVHTRGLD.

In some embodiments, the signal comprises phosphorylation of a residue within a signaling domain of said intracellular domain. In some embodiments, the signal induces activation of immune cell effector function. In some embodiments, the signal induces secretion of at least one cytokine from said modified cell. In some embodiments, the at least one cytokine is interleukin-2 (IL-2). In some embodiments, the signal induces activation of ZAP-70 kinase.

In some embodiments, the intracellular domain comprises an artificial amino acid sequence of sufficient length and charge to allow for detectable expression of the chimeric transmembrane polypeptide on a surface of the modified cell. In some embodiments, detection of the chimeric transmembrane polypeptide on a surface of the modified cell comprises FACS. In some embodiments, the intracellular domain comprises an artificial amino acid sequence of sufficient length and charge to allow for mobility of the chimeric transmembrane polypeptide within a membrane of said modified immune cell.

In some embodiments, the intracellular domain comprises an intracellular domain of any transmembrane protein other than CD3, CD28, OX-40 CD80, CD86 and a T-cell receptor (TCR). In some embodiments, the intracellular domain comprises CD3 Zeta chain mutated to be unable to transduce an activating signal.

In some embodiments, the CD3 Zeta chain comprises the amino acid sequence

(SEQ ID NO: 30)
RAKFSRSAETAANLQDPNQLYNELNLGRREEYDVLEKKRARDPE
MGGKQQRRRNPQEGVYNALQKDKMAEAYSEIGTKGERRRGKGHD
GLYQGLSTATKDTYDALHMQTLAPR.

In some embodiments, at least one tyrosine of said CD3 Zeta chain is mutated, and the mutation renders the intracellular domain unable to transduce an activating signal. In some embodiments, the at least one tyrosine is mutated to a phenylalanine. In some embodiments, all tyrosines of said CD3 Zeta chain have been mutated. In some embodiments, all tyrosines are mutated to phenylalanines.

In some embodiments, the intracellular domain comprises the amino acid sequence

(SEQ ID NO: 31)
RAKFSRSAETAANLQDPNQLFNELNLGRREEFDVLEKKRARDPE
MGGKQQRRRNPQEGVFNALQKDKMAEAFSEIGTKGERRRGKGHD
GLFQGLSTATKDTFDALHMQTLAPR.

In some embodiments, the intracellular domain is not capable of transducing any signal that renders the modified cell harmful to the target cell. In some embodiments, the intracellular domain is inert.

In some embodiments, the chimeric transmembrane polypeptide further comprises a tag. In some embodiments, the tag is selected from a GFP tag and a Myc tag.

In some embodiments, the target receptor is associated with a disease or disorder. In some embodiments, the target receptor is selected from GHR, GLP1R, TrkB, and PD-1.

In some embodiments, the target receptor is TrkB and the anti-TrkB antigen binding domain comprises an amino acid sequence with at least 70% identity to a sequence provided in any one of SEQ ID NO: 10, SEQ ID NO: 11, SEQ ID NO: 12, or SEQ ID NO: 13. In some embodiments, the chimeric transmembrane polypeptide comprises an amino acid sequence as set forth in SEQ ID NO: 18, or SEQ ID NO:20.

In some embodiments, the target receptor is GLP1R and the anti-GLP1R antigen binding domain comprises an amino acid sequence with at least 70% identity to a sequence provided in any one of SEQ ID NO: 14, or SEQ ID NO: 15. In some embodiments, the chimeric transmembrane polypeptide has the amino acid sequence as set forth in SEQ ID NO: 22.

In some embodiments, the target receptor is GHR and said target receptor-binding domain comprises a growth hormone (GH). In some embodiments, the GH comprises an amino acid sequence with at least 70% identity to the sequence provided in SEQ ID NO: 16. In some embodiments, the chimeric transmembrane polypeptide has the amino acid sequence as set forth in SEQ ID NO: 24.

In some embodiments, the target receptor is PD-1 and the anti-PD-1 antigen binding domain comprises an amino acid sequence with at least 70% identity to the sequence provided in SEQ ID NO: 25 or SEQ ID NO: 26. In some embodiments, the chimeric transmembrane polypeptide has the amino acid sequence as set forth in SEQ ID NO: 28.

In some embodiments, the chimeric transmembrane polypeptide has at least 95% sequence identity to an amino acid sequence of any one of SEQ ID NOs: 18, 20, 22, 24 or 28 and is not capable of transducing a signal.

In some embodiments, the modified cell or provided cell is an immune cell. In some embodiments, the immune cell is selected from a T-cell, a natural killer (NK) cell, a B-cell, a myeloid cell, a macrophage, a monocyte, a neutrophil, an antigen presenting cell and a dendritic cell.

In some embodiments, the modified cell or provided cell is derived from a primary human cell from a human donor, and wherein said modified primary human cell is suitable for use in human therapy. In some embodiments, the provided cell is autologous to the subject. In some embodiments, the provided cell is allogenic to the subject.

In some embodiments, the target cell is in culture. In some embodiments, the target cell is in a subject.

In some embodiments, the providing comprises extracting a primary cell from said subject.

In some embodiments, the activation of said target receptor on a cell of the subject treats the disease or disorder, and the extracellular receptor-binding domain comprises an agonist of the target receptor. In some embodiments, the inhibition of the receptor on a cell of the subject treats the disease or disorder, and the extracellular receptor-binding domain comprises an antagonist of the target receptor.

In some embodiments, the target receptor is TrkB and the disease or disorder is a neurological disease or disorder. In some embodiments, the neurological disease or disorder is selected from: Alzheimer's disease, depression, memory loss, amyotrophic lateral sclerosis (ALS), epilepsy and brain cancer.

In some embodiments, the target receptor is GLP1R and the disease or disorder is a metabolic or cardiovascular disease or disorder. In some embodiments, the metabolic disease or disorder is selected from: diabetes, obesity, glycogen storage disease, Parkinson's disease and mitochondrial myopathy. In some embodiments, the cardiovascular disease or disorder is selected from: stroke, myocardial infarction, cardiac ischemia, and coronary artery disease.

In some embodiments, the target receptor is GHR and the disease or disorder is a growth disease or disorder. In some embodiments, the growth disease or disorder is selected from: acromegaly, growth hormone deficiency, cancer, Turner syndrome, and Prader-Willi syndrome.

In some embodiments, the target receptor is PD-1 and the disease or disorder is an immune disease or disorder or cancer. In some embodiments, the immune disease or disorder is selected from: lupus, rheumatoid arthritis, psoriasis, Graves' disease, immune-mediated inflammation, and celiac disease. In some embodiments, the disease or disorder is cancer and the target receptor-binding domain comprises a PD-1 antagonist.

According to another aspect, there is provided a pharmaceutical composition, comprising a modified cell comprising a chimeric transmembrane polypeptide comprising at least one extracellular target receptor-binding domain, a transmembrane domain and an intracellular domain, wherein the intracellular domain does not transduce a signal and a pharmaceutically acceptable carrier, excipient, or adjuvant.

In some embodiments, the compositions of the invention are for use in treating a disease or disorder associated with the target receptor. In some embodiments, the composition comprises at least 1 million modified cells.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1A is photographic exposure of western-blots detecting pTrkB, total TrkB and actin.

FIG. 1B is a bar graph summarizing the relative amounts of pTrkb presented in FIG. 1A. Acting and total TrkB are used to standardize the expression of the phosphorylated form.

FIG. 2 is a bar graph showing the relative amount of IL-2 secreted from cultured cells. Results are shown as OD levels (650 wave length) from the IL-2 ELISA assay.

FIGS. 3A-3G are histograms of surface expression of Myc or GFP after viral transduction of (3A) αTrkB-Myc-WT, (3B) αTrkB-Myc-Mut, (3C) αTrkB-GFP-WT, (3D) αTrkB-GFP-Mut, (3E) αGLP1R-Myc-WT, and (3F) αGLP1R-Myc-Mut into BW cells and (3G) αGLP1R-Myc-Mut into primary murine T-helper cells. Light grey-unstained cells or non-transduced cells, grey—2nd antibody only control for Myc figures (anti-APC), black—Myc or GFP transduced cells.

FIG. 4 is a bar graph showing the relative amount of IL-2 secreted from transduced cells expressing Myc-containing constructs after co-incubation with anti-Myc antibody. Results are shown as OD levels (650 wave length) from the IL-2 ELISA assay.

FIG. 5 is a bar graph showing the relative amount of IL-2 secreted from transduced cells expressing Myc-containing constructs after co-incubation with GLP1R expressing cells. Results are shown as OD levels (650 wave length) from the IL-2 ELISA assay.

FIG. 6 is a bar graph showing the relative amount of TNF alpha secreted from Raw cells. Murine TNFα levels in the supernatant were determined by commercial ELISA kit. Results of ELISA assay are presented as OD-650.

### DETAILED DESCRIPTION OF THE INVENTION

The present invention provides compositions and methods for treatment of diseases by modulation of the activity of a receptor on a target cell. The present invention exploits the ability of cells of the immune system to migrate within the body and to accumulate at specific niches within organs. Such immune cells are used as a delivery platform for a molecule of interest that is expressed and anchored to their membrane. The anchored therapeutic molecule is in the extracellular domain of a chimeric transmembrane polypeptide, which comprises an intracellular domain that is not capable of transducing an immune cell activating signal. In this way biding of the therapeutic molecule to its target receptor will modulate (e.g., activate or inactive) that target receptor, but not activate the immune cell itself.

By a first aspect, there is provided a method of modulating signaling by a target receptor in a target cell, the method comprising contacting the target cell with a modified cell comprising a chimeric transmembrane polypeptide comprising at least one extracellular target receptor-binding domain, a transmembrane domain and an intracellular domain, wherein the intracellular domain is not capable of transducing any signal, thereby modulating signaling by a target receptor in a target cell.

By another aspect, there is provided a method of modulating signaling by a target receptor in a target cell, the method comprising contacting the target cell with a modified immune cell comprising a chimeric transmembrane polypeptide comprising at least one extracellular target receptor-binding domain, a transmembrane domain and an intracellular domain, wherein the intracellular domain is not capable of transducing an immune cell activating signal, thereby modulating signaling by a target receptor in a target cell.

By another aspect, there is provided a method of treating a subject suffering from a disease or disorder, the method comprising:
a. Providing an immune cell;
b. expressing in said immune cell a chimeric transmembrane polypeptide comprising at least one extracellular target receptor-binding domain, a transmembrane domain and an intracellular domain, wherein said intracellular domain is not capable of transducing an immune cell activating signal and wherein said target receptor is associated with said disease or disorder; and
c. administering said immune cell expressing said chimeric transmembrane polypeptide to said subject;
   thereby treating said subject suffering from a disease or disorder.

By another aspect, there is provided a method of treating a subject suffering from a disease or disorder, the method comprising:
a. Providing a cell capable of homing to a site of said disease or disorder;
b. expressing in said cell a chimeric transmembrane polypeptide comprising at least one extracellular target receptor-binding domain, a transmembrane domain and an intracellular domain, wherein said intracellular domain is inert and wherein said target receptor is associated with said disease or disorder; and
c. administering said cell expressing said chimeric transmembrane polypeptide to said subject;
   thereby treating said subject suffering from a disease or disorder.

By another aspect, there is provided a chimeric transmembrane polypeptide comprising at least one extracellular target receptor-binding domain, a transmembrane domain and an intracellular domain, wherein the intracellular domain is not capable of transducing an immune cell activating signal. In some embodiments, the intracellular domain is not capable of transducing any signal.

As used herein, the term “signaling” refers to intracellular signal transduction. Signal transduction is well known in the art, and refers to transmission of a signal through a series of molecular events to effect a physiological change in a cell. In some embodiments, the signaling comprises a signaling cascade within the target cell. In some embodiments, signal transduction comprises phosphorylation of signaling proteins. In some embodiments, inducing signaling comprises phosphorylation of a residue within a signaling domain of the target receptor. In some embodiments, inducing signaling comprises phosphorylation of a residue within a signaling domain of a protein of the signaling cascade. In some embodiments, the phosphorylated residue is a tyrosine residue. In some embodiments, inducing signaling comprises upregulation of a level of a downstream target of the target receptor. In some embodiments, inhibiting signaling comprises down-regulation of a level of a downstream target of the target receptor.

In some embodiments, inducing signaling comprises at least a 10%, 20%, 30%, 40%, 50%, 60%, 70%, 80%, 90%, or 100% increase in signaling. Each possibility represents a separate embodiment of the invention. In some embodiments, inhibiting signaling comprises at least a 10%, 20%, 30%, 40%, 50%, 60%, 70%, 80%, 90%, 95% or 100% reduction in signaling. Each possibility represents a separate embodiment of the invention.

Measuring changes in signaling is well known in the art and any method of confirming alterations in signaling may be used to perform the methods of the invention. Such methods of measuring include but are not limited to, measuring phosphorylation of a signaling domain of the target receptor or another signaling protein known to be down-stream of the receptor in a signaling cascade. Measuring expression levels of proteins known to be up-or down-regulated by signaling may also be employed. Example of such molecules include, but are not limited to cytokines, transcription factors, and effector proteins.

In some embodiments, the target cell is a cell of a diseased tissue. In some embodiments, the tissue is selected from: the brain, a muscle, the heart, the lungs, the pancreas, the skin, the liver, the reproductive system, the digestive system and the secretory system. In some embodiments, the target cell is a hematological cell. In some embodiments, the target cell is an immunological cell. In some embodiments, the target cell is selected from: a neuron, a muscle cell, a bone cell, a blood cell, a lymph cell, a fibroblast, an immune cell, an insulin-producing cell and a cardiac cell.

In some embodiments, the target cell is in culture. In some embodiments, the target cell is in a subject. In some embodiments, the target cell is in a subject and the modified cell is allogenic to the subject. In some embodiments, the target cell is in a subject and the modified cell is autologous to the subject. In some embodiments, the target cell is in a subject and the modified cell is further modified to not elicit an immune response or elicit a reduced immune response upon administration to the subject.

In some embodiments, the target receptor is a receptor associated with a disease or disorder. In some embodiments, the target receptor is on a diseased cell or diseased tissue of a subject. In some embodiments, activation of the target receptor treats or ameliorates the disease or disorder, and the extracellular receptor-binding domain comprises an agonist of the target receptor. In some embodiments, inhibition of the target receptor treats or ameliorates the disease or disorder, and the extracellular receptor-binding domain comprises an antagonist of the target receptor.

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

### Modified Cells

By another aspect, there is provided a modified cell comprising a chimeric transmembrane polypeptide comprising at least one extracellular target receptor-binding domain, a transmembrane domain and an intracellular domain, wherein the intracellular domain is not capable of transducing an immune cell activating signal. In some embodiments, the intracellular domain is not capable of transducing any signal. In some embodiments, the intracellular domain is inert. In some embodiments, the methods of the invention are performed by contacting a target cell with a modified cell of the invention. In some embodiments, the methods of the invention are performed by administering to a subject in need thereof, a modified cell of the invention.

In some embodiments, the modified cell of the invention is any cell which can home to a desired location or target cell in a body. In some embodiments, the modified cell of the invention is any cell which can home to a site of disease in the body. In some embodiments, the cells are fibroblasts. In some embodiments, the cells are mesenchymal stem cells (MSCs). In some embodiments, the cells are embryonic stem cells (ESCs) or induced pluripotent stem cells (IPSCs). In some embodiments, the cells are pluripotent stem cells (PSCs). In some embodiments, the cells are immune cells. In some embodiments, the immune cell is selected from a T-cell, a natural killer (NK) cell, a B-cell, a myeloid cell, a macrophage, a monocyte, a neutrophil, an antigen presenting cell and a dendritic cell. In some embodiments, the immune cell is a T-cell. In some embodiments, the T-cell is a CD4+ or a CD8+ T-cell. In some embodiment, the immune cell is partially activated to facilitate homing and synapse formation. In some embodiments, the immune cell is capable of crossing the blood brain barrier (BBB). In some embodiments, the immune cell is capable of crossing the blood testes barrier (BTB).

Cell homing may depend on the location or tissue of a target cell or diseased cell. In embodiments wherein the target cell is at a wound, the modified cell may be a fibroblast. In embodiments wherein the target cell is in the brain, the modified cell may be a CD4+ T-cell. In embodiments wherein the target cell is in the pancreas, the modified cell may be a B-cell.

Modified cells of the present invention are genetically modified to express and anchor to their membrane an agonist or an antagonist or the target receptor. In some embodiments, the agonist/antagonist is a single chain antibody which has agonistic/antagonistic properties for a target receptor, referred hereafter as single chain agonist/antagonist antibody (SCAAB). The cells of the present invention are used as carrier cells which mediate the delivery of the agonist/antagonist to the desired target site in the body of a subject. In some embodiments, the carrier cells are introduced to the body of a subject where they migrate and accumulate at a desired organ and/or at a specific niche. It will be understood to one skilled in the art, that as the cells are carriers of the agonist and/or antagonist, cytotoxic activation of the modified cell is not required for the methods of the invention. Indeed, as activation of immune cell effector function initiates an immune response and/or renders the cell cytotoxic, activation of effector function in the cell is disadvantageous and not desired.

In some embodiments, the methods of the invention seek to activate or inhibit a target receptor on a target cell, and do not wish to kill the target cell as is likely to result if the immune cell becomes fully activated upon binding. As such the chimeric transmembrane polypeptides of the invention have intracellular regions that are not capable of transducing an immune cell activation signal, leading to harming target cells, and thus immune cells, such as CD8 T cells and NK cells, are not rendered cytotoxic upon binding to the target. In some embodiments, the modified immune cells of the invention are not cytotoxic. In some embodiments, the modified immune cells of the invention are not rendered cytotoxic by binding to the target receptor. In some embodiments, the modified immune cells of the invention do not secrete cytokines as a result of binding to the target receptor. In some embodiments, the modified cells do not harm the target cell. In some embodiments, the modified cells do not kill the target cells. In some embodiments, the modified cells are not detrimental to the target cell.

In some embodiments, in which the modified cell is an immune cell the cytoplasmic domain is not capable of transducing an immune cell activating signal. In some embodiments, in which the modified cell is an immune cell the cytoplasmic domain is not capable of transducing any signal into the cell that would result is the modified cell harming the target cell. In some embodiments, in which the modified cell is an immune cell the cytoplasmic domain is not capable of transducing any signal into the cell. In some embodiments, in which the modified cell is another homing cell other than an immune cell the cytoplasmic domain is not capable of transducing any signal into the cell that would result in the modified cell harming the target cell. In some embodiments, in which the modified cell is another homing cell other than an immune cell the cytoplasmic domain is not capable of transducing any signal into the cell.

In some embodiments, the modified cell is a primary cell. In some embodiments, the modified cell is derived from a primary cell from a human donor, and wherein said modified primary human cell is suitable for use in human therapy. In some embodiments, the primary cell is cultured in cell culture media after removal from the human donor. In some embodiments, the modified cell is a daughter or descendant cell from the primary cell put in culture. Culturing primary cells for return to the donor is well known in the art, any culture condition that can be used to keep the cell healthy suitable for return to the donor is suitable. Instructions for primary cell culture can be obtained from ATCC, Sigma Aldrich as two non-limiting examples. In some embodiments, the primary cell is expanded in culture and the expanded modified cells are returned to the donor. In some embodiments, the providing comprises extracting a primary immune cell from the patient.

The term “cell culture medium” refers to any liquid medium which enables cells proliferation. Growth media are known in the art and can be selected depending of the type of cell to be grown. For example, a growth medium for use in growing mammalian cells is Dulbecco's Modified Eagle Medium (DMEM) which can be supplemented with heat inactivated fetal bovine serum.

In some embodiments, modified cells are cultured under effective conditions, which allow for the expression of high amounts of chimeric polypeptide. In some embodiments, effective culture conditions include, but are not limited to, effective media, bioreactor, temperature, pH and oxygen conditions that permit protein production. In one embodiment, an effective medium refers to any medium in which a cell is cultured to produce the chimeric polypeptide of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid.

As used herein, the term “chimeric” polypeptide refers to a polypeptide in which at least two fragments from distinct naturally occurring proteins have been combined into a single non-naturally occurring protein. Chimeric proteins are well known to one skilled in the art, and will have a distinct domain or fragment which can be identified as being derived for a known protein and at least one other distinct domain or fragment which can be identified as being derived from a second known protein. In some embodiments, a naturally occurring protein is not a chimeric protein. In some embodiments, a naturally occurring protein with a leader sequence or peptide from a different protein is not considered a chimeric peptide. It will be understood that as the leader sequence or peptide is not expressed in the mature protein, it cannot be considered to contribute to the polypeptide being chimeric. In some embodiments, the chimeric transmembrane polypeptide comprises an extracellular domain for a first protein and an intracellular domain from a second protein.

As used herein, a “fragment” refers to a portion of a protein which is of sufficient size or structure so as to still be recognizable as part of the whole protein. In some embodiments, a fragment is the entire protein. As used herein, the term “derived from” or “corresponding to” refers to construction of an amino acid sequence based on the knowledge of a sequence using any one of the suitable means known to one skilled in the art, e.g. chemical synthesis in accordance with standard protocols in the art.

As used herein, a “leader sequence” or “leader peptide” are synonymous and refer to the 15-30 mostly hydrophobic amino acids found that the N-terminus of proteins which are inserted into a membrane. Most commonly the leader sequence is cleaved from the mature protein after insertion into a membrane. These sequences are often required for proper insertion into the endoplasmic reticulum, correct orientation of transmembrane proteins and robust expression on the cell surface. Generally, the leader sequence from any transmembrane protein that is expressed on the cell surface may be exchanged with the leader sequence from any other transmembrane protein expressed on the cell surface and robust surface expression is still achieved. Leader peptides from proteins predominantly expressed intracellularly, such as mitochondrial proteins for example, should not ideally be used, but if surface expression is achieved are still optional. Examples of leader sequences which may be used for insertion of a chimeric transmembrane polypeptide of the invention into a cell membrane, and thus efficient cell surface expression, include but are not limited to, MDMRVPAQLLGLLLLWLSGARC (SEQ ID NO: 1), MDMRVPAQLLGLLLLWLSGARCQ (SEQ ID NO: 2), and MATGSRTSLLLAFGLLCLPWLQEGSQA (SEQ ID NO: 3).

As used herein, a “transmembrane polypeptide” refers to a protein that is anchored in and spans the plasma membrane. As such a transmembrane polypeptide must comprise at least extracellular amino acid, at least one intracellular amino acid and amino acid sequence which is within the plasma membrane. Transmembrane proteins are well known in the art, and it will be understood to one skilled in the art that a GPI anchored polypeptide is not a polypeptide of the invention as it is not “transmembrane” and does not have an intracellular domain.

In some embodiments, the modified cell of the invention comprises more than one chimeric transmembrane polypeptide. In some embodiments, a single chimeric transmembrane polypeptide comprises more than one target receptor-binding domain. In some embodiments, more than one target receptor-binding domain target the same receptor. In some embodiments, two distinct receptor-binding domains target different receptors. As such a single modified cell can have more than one target receptor and more than one target cell.

### Extracellular Domain

In some embodiments, the extracellular domain comprises an agonist or antagonist of the target receptor. In some embodiments, the target receptor-binding domain comprises an antibody's antigen binding domain. In some embodiments, the extracellular domain contains more than one target receptor-binding domain. In some embodiments, the extracellular domain may bind to more than one target receptor. In some embodiments, the extracellular domain may comprise more than one agonist or antagonist for the target receptor. In some embodiments, the target receptor-binding domain comprises an immunoglobulin variable heavy chain domain (VH) and an immunoglobulin variable light chain domain (VL). In some embodiments, the extracellular domain comprises more than one antibody antigen binding domain. As the methods of the invention are for modulating a target receptor, it will be understood that extracellular domains that merely bind a target receptor but do not activate or inhibit that receptor are not embodiments of the invention. Further, target receptor-binding domains merely used for targeting of the modified cell to a target cell also would not modulate target receptor signaling and also are not embodiments of the invention.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

In some embodiments, the VH and VL are connected by a peptide linker. In the design of antigen binding fragments for use as antibodies peptide linkers between the VH and VL are well known, and any linker effective in generating a binding pocket that successfully binds to the target receptor may be used. In some embodiments, the peptide linker comprises the amino acid sequence GGSSRSSSSGGGGSGGGG (SEQ ID NO: 4) or GGGGSGGGGSGGGGSGGGGS (SEQ ID NO: 5).

In some embodiments, the target receptor-binding domain binds the target receptor with a dissociation constant (Kd) of at least 0.1, 0.2, 0.3, 0.4, 0.5, 1, 5, 10, 20, 30, 40, 50, 60, 70, 80, 90, or 100 pM. Each possibility represents a separate embodiment of the invention. In some embodiments, the target receptor-binding domain binds the target receptor with a Kd of between 0.1 and 100, 0.1 and 200, 0.1 and 300, 0.1 and 400, 0.1 and 500, 0.1 and 600, 0.1 and 700, 0.1 and 800, 0.1 and 900, 0.1 and 1000, 0.1 and 2000, 0.1 and 3000, 0.5 and 100, 0.5 and 200, 0.5 and 300, 0.5 and 400, 0.5 and 500, 0.5 and 600, 0.5 and 700, 0.5 and 800, 0.5 and 900, 0.5 and 1000, 0.5 and 2000, 0.5 and 3000, 1 and 100, 1 and 200, 1 and 300, 1 and 400, 1 and 500, 1 and 600, 1 and 700, 1 and 800, 1 and 900, 1 and 1000, 1 and 2000, or 1 and 3000 pM. Each possibility represents a separate embodiment of the invention.

In some embodiments, the target receptor-binding domain binds the target receptor with a half maximal effective concentration (EC50) of at least 0.1, 0.2, 0.3, 0.4, 0.5, 1, 5, 10, 20, 30, 40, 50, 60, 70, 80, 90, or 100 nM. Each possibility represents a separate embodiment of the invention. In some embodiments, the target receptor-binding domain binds the target receptor with a EC50 of between 0.1 and 100, 0.1 and 200, 0.1 and 300, 0.1 and 400, 0.1 and 500, 0.1 and 600, 0.1 and 700, 0.1 and 800, 0.1 and 900, 0.1 and 1000, 0.1 and 2000, 0.1 and 3000, 0.5 and 100, 0.5 and 200, 0.5 and 300, 0.5 and 400, 0.5 and 500, 0.5 and 600, 0.5 and 700, 0.5 and 800, 0.5 and 900, 0.5 and 1000, 0.5 and 2000, 0.5 and 3000, 1 and 100, 1 and 200, 1 and 300, 1 and 400, 1 and 500, 1 and 600, 1 and 700, 1 and 800, 1 and 900, 1 and 1000, 1 and 2000, or 1 and 3000 nM. Each possibility represents a separate embodiment of the invention.

### Transmembrane Domain

Transmembrane domains are well known in the art and generally contain hydrophobic residues. In some embodiments, the transmembrane domain is a known transmembrane domain from a naturally occurring protein. In some embodiments, the transmembrane domain is from a murine transmembrane protein. In some embodiments, the transmembrane domain is from a human transmembrane protein. In some embodiments, the transmembrane domain is a synthetic transmembrane domain. The methods and compositions of the invention may be performed with any transmembrane domain that results in anchorage of the chimeric polypeptide of the invention in the plasma membrane with the target receptor-binding domain in the extracellular region and the intracellular region lacking the capability of transducing an immune cell activating signal.

In some embodiments, the transmembrane domain is a single-pass transmembrane domain. In some embodiments, the transmembrane domain comprises an odd number of transmembrane regions. Once skilled in the art will understand that if an even number of transmembrane regions are present in the protein than the intracellular region will be an intracellular loop found between two transmembrane regions. In some embodiments, the intracellular region is at the C-terminus of the protein. In some embodiments, the intracellular region is at the N-terminus of the protein. In some embodiments, the intracellular region is an intracellular loop between two transmembrane domains.

In some embodiments, the transmembrane domain comprises the CD3 transmembrane domain. In some embodiments, the transmembrane domain consists of the CD3 transmembrane domain. In some embodiments, the CD3 transmembrane domain comprises the sequence LCYLLDGILFIYGVIITALYL (SEQ ID NO: 29). In some embodiments, the CD3 transmembrane domain consists of the sequence provided in SEQ ID NO: 29.

In some embodiments, the chimeric transmembrane polypeptide further comprises a hinge region. In some embodiments, the hinge region is an extracellular region. In some embodiments, the hinge region is membrane proximal. In some embodiments, the hinge region is an extracellular and membrane proximal region. In some embodiments, the hinge region comprises an immunoglobulin hinge region. Use of a hinge region in antibodies and chimeric proteins is well known in the art. In some embodiments, the hinge region links the VH and VL domains. In some embodiments, only polypeptides comprising VH and VL domains comprise a hinge domain.

In some embodiments, the hinge region comprises a CD-8 hinge region. In some embodiments, the hinge region consists of a CD-8 hinge region. In some embodiments, the CD-8 hinge region comprises the amino acid sequence ALSNSIMYFSHFVPVFLPAKPTTTPAPRPPTPAPTIASQPLSLRPEASRPAAGGAV HTRGLD (SEQ ID NO: 7). In some embodiments, the CD-8 hinge region consists of the amino acid sequence provided in SEQ ID NO: 7.

### Intracellular Domain

In some embodiments, the intracellular domain of the chimeric transmembrane polypeptide of the invention is not capable of transducing an immune cell activating signal. In some embodiments, the intracellular domain of the chimeric transmembrane polypeptide of the invention is not capable of transducing any signal that results in the modified cell harming the target cell. In some embodiments, the intracellular domain of the chimeric transmembrane polypeptide of the invention is not capable of transducing any signal. In some embodiments, the intracellular domain of the chimeric transmembrane polypeptide of the invention is inert. As used herein, the term “inert” refers to the inability to the protein region to affect signaling in any way. An inert protein domain not only cannot transduce a signal, but it cannot regulate or modulate a signal. An inert cytoplasmic domain may still affect the proteins ability to migrate within the membrane or affect the proteins expression or orientation in the membrane.

In some embodiments, immune cell activation comprises effector function activation. In some embodiments, immune cell activation comprises at least one of secretion of cytokines, release of antibodies, activation of ZAP-10 kinase, immune cell proliferation and upregulation of surface expression at least one of CD69, CD71, CD25, HLA-DR and CTLA-4. In some embodiments, immune cell activation comprises secretion of IL-2. In some embodiments, an immune cell activating signal induces activation of ZAP-70 kinase. In some embodiments, an immune cell activating signal comprises phosphorylation of a residue within a signaling domain of an intracellular domain of a transmembrane protein. In some embodiments, the residue is a tyrosine residue. In some embodiments, an immune cell activating signal comprises activation of effector function. In some embodiments, an immune cell activating signal comprises phosphorylation of a tyrosine within a signaling domain of an intracellular domain of a protein of the T-cell receptor complex or a costimulatory protein. In some embodiments, the costimulatory protein is any one of CD28, OX-40, CD80 (B7-1) and CD86 (B7-2). In some embodiments, an immune cell activating signal comprises co-stimulation of the TCR by any one of CD28, OX-40, CD80 and CD86.

In some embodiments, transducing an immune cell activating signal comprises costimulatory signaling. In some embodiments, transducing an immune cell activating signal comprises any function that absent this function the signal would not be transduced, and the cell not activated. In some embodiments, transducing the signal comprises a kinase cascade. In some embodiments, transducing the signal comprises protein-protein binding of kinase molecules and costimulatory molecules.

In some embodiments, a cytoplasmic domain capable of transducing an immune cell activating signal comprises an immunoreceptor tyrosine-based activation motif (ITAM) domain. Immune cell activating receptors are well known in the art. Non-limiting examples of immune cell activating proteins, who comprise an ITAM domain, and whose signaling domains cannot be used in the cytoplasmic domain of the chimeric polypeptide of the invention without mutation to abolish signaling are provided in Table 1.

TABLE 1

| Symbol | Description | Gcid |
|---|---|---|
| SYK | Spleen Associated Tyrosine Kinase | GC09P090831 |
| NFAM1 | NFAT Activating Protein with ITAM | GC22M042380 |
| | Motif 1 | |
| STAM2 | Signal Transducing Adaptor | GC02M152116 |
| | Molecule 2 | |
| STAM | Signal Transducing Adaptor Molecule | GC10P017645 |
| ZAP70 | Zeta Chain Of T-Cell Receptor | GC02P097696 |
| | Associated Protein Kinase 70 | |
| TYROBP | TYRO Protein Tyrosine Kinase | GC19M035904 |
| | Binding Protein | |
| LCK | LCK Proto-Oncogene, Src Family | GC01P032251 |
| | Tyrosine Kinase | |
| FLNA | Filamin A | GC0XM154348 |
| CD79A | CD79a Molecule | GC19P041877 |
| CD247 | CD247 Molecule | GC01M167399 |
| CARD9 | Caspase Recruitment Domain | GC09M136361 |
| | Family Member 9 | |
| FCER1G | Fc Fragment of IgE Receptor Ig | GC01P161215 |
| CLEC4E | C-Type Lectin Domain Family 4 | GC12M008535 |
| | Member E | |
| CD79B | CD79b Molecule | GC17M063928 |
| FCGR2A | Fc Fragment of IgG Receptor IIa | GC01P161505 |
| CD3E | CD3e Molecule | GC11P118304 |
| PIK3CB | Phosphatidylinositol-4,5-Bisphosphate | GC03M138652 |
| | 3-Kinase Catalytic Subunit Beta | |
| RASGRP1 | RAS Guanyl Releasing Protein 1 | GC15M038488 |
| CD300LB | CD300 Molecule Like Family | GC17M074520 |
| | Member B | |
| PILRB | Paired Immunoglobin-Like Type 2 | GC07P100353 |
| | Receptor Beta | |
| GCSAM | Germinal Center Associated | GC03M112120 |
| | Signaling and Motility | |
| BCR | BCR, RhoGEF And GTPase | GC22P023179 |
| | Activating Protein | |
| CEACAM4 | Carcinoembryonic Antigen Related | GC19M041618 |
| | Cell Adhesion Molecule 4 | |
| GP6 | Glycoprotein VI Platelet | GC19M055013 |
| GRB2 | Growth Factor Receptor Bound | GC17M075318 |
| | Protein 2 | |
| SHC1 | SHC Adaptor Protein 1 | GC01M154962 |
| EZR | Ezrin | GC06M158765 |
| FCAR | Fc Fragment of IgA Receptor | GC19P054983 |
| CD300LD | CD300 Molecule Like Family | GC17M074579 |
| | Member D | |
| LYN | LYN Proto-Oncogene, Src Family | GC08P055879 |
| | Tyrosine Kinase | |
| PLCG2 | Phospholipase C Gamma 2 | GC16P081773 |
| OSCAR | Osteoclast Associated, Immuno- | GC19M054094 |
| | globulin-Like Receptor | |
| CD3D | CD3d Molecule | GC11M118338 |
| CD3G | CD3g Molecule | GC11P118344 |
| RHOH | Ras Homolog Family Member H | GC04P040192 |
| FCGR2C | Fc Fragment of IgG Receptor IIc | GC01P161551 |
| | (Gene/Pseudogene) | |
| AKT1 | AKT Serine/Threonine Kinase 1 | GC14M104769 |
| BTK | Bruton Tyrosine Kinase | GC0XM101349 |
| STAT1 | Signal Transducer and Activator of | GC02M190964 |
| | Transcription 1 | |
| EGF | Epidermal Growth Factor | GC04P109912 |
| ITGB2 | Integrin Subunit Beta 2 | GC21M044885 |
| CHUK | Conserved Helix-Loop-Helix | GC10M100188 |
| | Ubiquitous Kinase | |
| TLR2 | Toll Like Receptor 2 | GC04P153684 |
| ICAM1 | Intercellular Adhesion Molecule 1 | GC19P010270 |
| FYN | FYN Proto-Oncogene, Src Family | GC06M111660 |
| | Tyrosine Kinase | |
| PIK3R1 | Phosphoinositide-3-Kinase | GC05P068215 |
| | Regulatory Subunit 1 | |
| ITGA2B | Integrin Subunit Alpha 2b | GC17M044373 |
| ITGB3 | Integrin Subunit Beta 3 | GC17P047254 |
| ITGAV | Integrin Subunit Alpha V | GC02P186589 |
| KRT18 | Keratin 18 | GC12P052948 |
| PTK2B | Protein Tyrosine Kinase 2 Beta | GC08P027311 |
| CREB1 | CAMP Responsive Element | GC02P207529 |
| | Binding Protein 1 | |
| RDX | Radixin | GC11M110134 |
| STAT2 | Signal Transducer and Activator | GC12M056341 |
| | of Transcription 2 | |
| NR4A1 | Nuclear Receptor Subfamily 4 | GC12P052022 |
| | Group A Member 1 | |
| FCGR2B | Fc Fragment of IgG Receptor IIb | GC01P161663 |
| BLNK | B-Cell Linker | GC10M096191 |
| NLRP3 | NLR Family Pyrin Domain | GC01P247415 |
| | Containing 3 | |
| NEDD4 | Neural Precursor Cell Expressed, | GC15M055826 |
| | Developmentally Down- | |
| | Regulated 4, E3 Ubiquitin Protein | |
| | Ligase | |
| MSN | Moesin | GC0XP065588 |
| MS4A1 | Membrane Spanning 4-Domains A1 | GC11P060474 |
| LAT | Linker for Activation Of T-Cells | GC16P028998 |
| INPP5D | Inositol Polyphosphate-5- | GC02P233059 |
| | Phosphatase D | |
| IL18R1 | Interleukin 18 Receptor 1 | GC02P102345 |
| LCP2 | Lymphocyte Cytosolic Protein 2 | GC05M170246 |
| S100A8 | S100 Calcium Binding Protein A8 | GC01M153391 |
| PTPRT | Protein Tyrosine Phosphatase, | GC20M042072 |
| | Receptor Type T | |
| SELPLG | Selectin P Ligand | GC12M108621 |
| CD300A | CD300a Molecule | GC17P074466 |
| CD244 | CD244 Molecule | GC01M160799 |
| SLAMF6 | SLAM Family Member 6 | GC01M160454 |
| ETV5 | ETS Variant 5 | GC03M186046 |
| IFNA1 | Interferon Alpha 1 | GC09P021441 |
| NCR2 | Natural Cytotoxicity Triggering | GC06P041303 |
| | Receptor 2 | |
| RASA2 | RAS P21 Protein Activator 2 | GC03P141487 |
| LAT2 | Linker for Activation Of T-Cells | GC07P074199 |
| | Family Member 2 | |
| CD300C | CD300c Molecule | GC17M074544 |
| CLEC1B | C-Type Lectin Domain Family 1 | GC12M011530 |
| | Member B | |
| FCRL3 | Fc Receptor Like 3 | GC01M157674 |
| FHOD1 | Formin Homology 2 Domain | GC16M067263 |
| | Containing 1 | |
| TNIP3 | TNFAIP3 Interacting Protein 3 | GC04M121131 |
| PILRA | Paired Immunoglobin Like Type 2 | GC07P100367 |
| | Receptor Alpha | |
| KLRF1 | Killer Cell Lectin Like Receptor F1 | GC12P009827 |
| SH2D4B | SH2 Domain Containing 4B | GC10P080914 |
| TARM1 | T-Cell-Interacting, Activating | GC19M054069 |
| | Receptor on Myeloid Cells 1 | |

In some embodiments, the intracellular domain comprises an intracellular domain of a transmembrane protein not expressed in immune cells. In some embodiments, the intracellular domain comprises an intracellular domain of a transmembrane protein expressed in immune cells, but not involved in cellular activation. In some embodiments, the intracellular domain comprises an intracellular domain capable of transducing an immune cell activation signal which has been mutated to be unable to transduce this signal. In some embodiments, the intracellular domain comprises an artificial amino acid sequence.

One skilled in the art will appreciate that the intracellular domain has other functions beyond signaling. The length, charge, and amino acid motifs of the intracellular domain can modulate proper insertion in the membrane, surface expression, protein folding, protein recycling and longevity, and mobility with the membrane among other aspects of the proteins and membranes dynamics.

In some embodiments, the intracellular domain comprises at least 1, 3, 5, 10, 15, 20, 25, 30, 35, 40, 45, 50, 75 or 100 amino acids. Each possibility represents a separate embodiment of the invention. In some embodiments, the intracellular domain comprises at most 10, 15, 20, 25, 30, 40, 45, 50, 75, 100, 150, 200, 250, 300, 350, 400, 450 or 500 amino acids. Each possibility represents a separate embodiment of the invention.

In some embodiments, the intracellular domain comprises at least one positively charged amino acid. In some embodiments, the intracellular domain comprises at least one positively charged amino acid within 5 amino acids from the end of the transmembrane domain. Positively charged amino acids include lysine (K), arginine (R) and histidine (H), as well as positively charged synthetic amino acids. One skilled in the art will appreciate that positive charge may aid in proper orientation of the polypeptide within the membrane and robust surface expression.

In some embodiments, the intracellular domain comprises an amino acid sequence of sufficient length and charge to allow for detectable expression of the chimeric transmembrane polypeptide on a surface of said modified cell. In some embodiments, detection of the chimeric transmembrane polypeptide on a surface of the modified cell comprises FACS. FACS is a well-known technique, and so long as no cell-permeabilization step is included, can be used to confirm surface expression of a protein. In some embodiments, the chimeric transmembrane polypeptide of the invention further comprises a tag. In some embodiments, the tag is in the extracellular region. In some embodiments, the tag is in the extracellular region and an antibody against the tag is used to confirm surface expression. In some embodiments, the tag is in the intracellular region. The tag may be positioned anywhere in the chimeric protein such that it does not interfere with receptor binding, does not interfere with robust surface expression and does not interfere with the mobility of the protein within the plasma membrane. In some embodiments, the tag is selected from a Myc tag and a fluorescent tag. In some embodiments, a Myc-tag comprises the amino acid sequence EQKLISEEDL (SEQ ID NO: 6). In some embodiments, the fluorescent tag is GFP. In some embodiments, the GFP tag is eGFP. In some embodiments, the GFP tag comprises the amino acid sequence

(SEQ ID NO: 33)
MVSKGEELFTGVVPILVELDGDVNGHKFSVSGEGEGDATYGKLT
LKFICTTGKLPVPWPTLVTTLTYGVQCFSRYPDHMKQHDFFKSA
MPEGYVQERTIFFKDDGNYKTRAEVKFEGDTLVNRIELKGIDFK
EDGNILGHKLEYNYNSHNVYIMADKQKNGIKVNFKIRHNIEDGS
VQLADHYQQNTPIGDGPVLLPDNHYLSTQSALSKDPNEKRDHMV
LLEFVTAAGITLGMDELYK.

In some embodiments, the intracellular domain comprises an amino acid sequence of sufficient length and charge to allow for mobility of the chimeric transmembrane polypeptide within a membrane of the modified cell. Lateral movement of a protein within a membrane can be regulated by many factors, among them the cytoplasmic region of the protein. Activation of the target receptor requires clustering of the chimeric protein at the synapse with the target cell. Thus, activation can be used to confirm that a chimeric protein is capable of lateral movement. Further tagged proteins may be visualized in vivo by the tag if the tag is fluorescent. Live field microscopy, and live cell imaging can be used to confirm lateral movement of the chimeric polypeptide within the membrane.

In some embodiments, the intracellular domain does not comprise an endoplasmic reticulum (ER) retention signal or mitochondrial-targeting motif. ER-retention signals are short motifs, usually only 3 amino acids long and comprising lysines and arginines, that cause a transmembrane protein to be retained in the ER and not be expressed on the cell surface. Mitochondrial targeting motifs are short amino acid sequences that target a protein to the mitochondria and not the plasma membrane. As the polypeptides of the invention must be expressed on the cell surface, motifs and signals that retain or target the polypeptide to any subcellular location or organelle other than the plasma membrane, should be avoided, although so long as some surface expression is achieved the intracellular domain may be used.

In some embodiments, the intracellular domain comprises an unmodified intracellular domain of any protein other than a protein that is capable of transducing an immune cell activation signal. In some embodiments, the intracellular domain comprises an unmodified intracellular domain of any protein other than CD3, CD28, OX-40 CD80, CD86 and a T-cell receptor (TCR). In some embodiments, the intracellular domain comprises an unmodified intracellular domain of any protein other than CD3, CD28, OX-40, CD80, and CD86. In some embodiments, the intracellular domain comprises an unmodified intracellular domain of any protein other than CD3. In some embodiments, the intracellular domain comprises an unmodified intracellular domain of any protein other than CD3 and CD80. In some embodiments, the intracellular domain comprises an unmodified intracellular domain of any protein other than CD3 and CD28. In some embodiments, the intracellular domain comprises an unmodified intracellular domain of any protein other than CD3, CD28 and CD80.

In some embodiments, the intracellular domain comprises a mutated intracellular domain of any protein capable of transducing an immune cell activation signal, wherein said mutating renders the intracellular domain unable to transduce an activating signal. In some embodiments, a mutated intracellular domain comprises mutation of at least one tyrosine residue of a signaling domain. In some embodiments, the signaling domain is an ITAM domain. In some embodiments, all tyrosines residues of the intracellular domain are mutated. In some embodiments, the tyrosine is mutated to an amino acid that cannot be phosphorylated. In some embodiments, the tyrosine is mutated to phenylalanine.

In some embodiments, the intracellular domain comprises a CD3 zeta chain mutated to be unable to transduce an activating signal. In some embodiments, CD3 Zeta chain comprises the amino acid sequence RAKFSRSAETAANLQDPNQLYNELNLGRREEYDVLEKKRARDPEMGGKQQRR RNPQEGVYNALQKDKMAEAYSEIGTKGERRRGKGHDGLYQGLSTATKDTYDA LHMQTLAPR (SEQ ID NO: 30). In some embodiments, mutated CD3 zeta chain comprises the amino acid sequence

(SEQ ID NO: 31)
RAKFSRSAETAANLQDPNQLFNELNLGRREEFDVLEKKRARDPE
MGGKQQRRRNPQEGVFNALQKDKMAEAFSEIGTKGERRRGKGHD
GLFQGLSTATKDTFDALHMQTLAPR.

In some embodiments, the intracellular domain comprises a CD80 (B7-1) cytoplasmic tail mutated to be unable to transduce an activating signal. In some embodiments, the intracellular domain comprises a CD80 (B7-1) cytoplasmic tail mutated to be unable to co-stimulate the TCR. In some embodiments, the CD80cytoplasmic tail comprises the amino acid sequence KCFCKHRSCFRRNEASRETNNSLTFGPEELALAEQTVFL (SEQ ID NO: 32). In some embodiments, mutated CD80 cytoplasmic tail comprises mutation of the RRNE motif at amino acids 11-14 of SEQ ID NO: 32. In some embodiments, the RRNE is mutated to AAAA. In some embodiments, mutated CD80 cytoplasmic tail comprises mutation of at least one serine of the cytoplasmic tail. In some embodiments, the serine is mutated to an alanine. In some embodiments, the serine at position 16 of SEQ ID NO: 32 is mutated. In some embodiments, the serine at position 22 of SEQ ID NO: 32 is mutated. In some embodiments the serine at position 16 and the serine at position 22 or SEQ ID NO: 32 are mutated. In some embodiments, the RRNE motif and at least one serine are mutated.

In some embodiments, the intracellular domain is inert. As used herein, an “inert” domain is not capable of any signaling. In some embodiments, an inert domain has no function. In some embodiments, the intracellular domain is not capable of any signaling. In some embodiments, the intracellular domain has been mutated so as to abrogate any signaling capability. In some embodiments, the intracellular domain is incapable of contributing to immune cell activation in any way.

The term “embryonic stem cell” refers to stem cells derived from the undifferentiated inner mass of a human embryo. Such cells are pluripotent, and capable of differentiating, or being differentiated by means known to one ordinary in the art, into cells of any lineage. In order for a hESC to be considered undifferentiated, it must continue to express stem cell markers or not express markers of differentiated cells.

ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hESI (MizMedi Hospital-Seoul National University); HSF-1, HSF-6 (University of California at San Francisco); and HI, H7, H9, HI 3, H14 (Wisconsin Alumni Research Foundation (WiCell Research Institute)). Stem cells of interest also include embryonic stem cells from other primates, such as Rhesus stem cells and marmoset stem cells. The stem cells may be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. No. 7,029,913, U.S. Pat. No. 5,843,780, and U.S. Pat. No. 6,200,806. Methods for proliferating hESCs in the undifferentiated form are described in WO 99/20741, WO 01/51616, and WO 03/020920.

By “induced pluripotent stem cell” or “iPSC” it is meant a pluripotent stem cell (PSC) that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERT, and zfp42. Examples of methods of generating and characterizing iPSCs may be found in, for example, U.S. Patent Publication Nos. US20090047263, US20090068742, US20090191159, US20090227032, US20090246875, and US20090304646. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

The term PSC refers to pluripotent stem cells regardless of their derivation, the term PSC encompasses the terms ESC and iPSC, as well as the term embryonic germ stem cells (EGSC), which are another example of a PSC. By “embryonic germ stem cell” (EGSC) or “embryonic germ cell” or “EG cell” is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235. PSCs may be in the form of an established cell line, they may be obtained directly from primary embryonic tissue, or they may be derived from a somatic cell. PSCs can be the modified cells of the methods described herein.

### Target Receptors

In some embodiments, the target receptor is selected from: Growth Hormone Receptor (GHR), Glucagon-like peptide 1 receptor (GLP1R), Tyrosine receptor kinase B/Tropomyosin receptor kinase B (TrkB), and Programmed cell Death protein 1 (PD-1).

In some embodiments, the target receptor is TrkB and the target receptor-binding domain comprises a VL comprising the amino acid sequence DVVMTQLPLSLPVILGDQASISCRSSQSLIHSNGNTYLHWYLQKPGQSPKLLIYK VSNRFSGVPDRFSGSGSGTDFTLKISRVEAEDLGVYFCSQSTHVPFTFGSGTKLEI KRA (SEQ ID NO: 10). In some embodiments, the target receptor is TrkB and the target receptor-binding domain comprises a VH comprising the amino acid sequence QVQLQQSGPELVKPGASVKLSCKASGYTFTSYDINWVKQRPGQGLEWIGWIYP RDGSIKFNEKFKGKATLTVDTSSSTAYMELHSLTSEDSAAYFCARRGRLLLYGF AYWGQGTLVTVSA (SEQ ID NO: 11). In some embodiments, the target receptor is TrkB and the target receptor-binding domain comprises SEQ ID NO:10 and SEQ ID NO: 11. In some embodiments, the target receptor is TrkB and the target receptor-binding domain comprises a VL comprising the amino acid sequence DVVMTQTPLSLPVSLGDQASISCRSSQSLVHSNGNTYLHWYLQKPGQSPNLLIY KVSNRFSGVPDRFSGSGSGTDFTLKISRVEAEDLGVYFCSQGTHVPYTFGGGTK LEIKRA (SEQ ID NO: 12). In some embodiments, the target receptor is TrkB and the target receptor-binding domain comprises a VH comprising the amino acid sequence QVQLQQSGAELVRPGASVTLSCKASGYTFTDYEMHWVKQTPVHGLEWIGTIDP ETAGTAYNNQKFKGKAILTAGKSSSTAYMELRSLTSEDSAVYYCTGVTTWFAY WGQGTLVTVSA (SEQ ID NO: 13). In some embodiments, the target receptor is TrkB and the target receptor-binding domain comprises SEQ ID NO: 12 and SEQ ID NO: 13.

In some embodiments, the target receptor is TrkB and the target receptor-binding domain comprises an analog of any one of SEQ ID NO: 10, SEQ ID NO: 11, SEQ ID NO: 12 and SEQ ID NO: 13. In some embodiments, the target receptor is TrkB and the target receptor-binding domain comprises a homolog of any one of SEQ ID NO: 10, SEQ ID NO: 11, SEQ ID NO: 12 and SEQ ID NO: 13. In some embodiments, the target receptor is TrkB and the target receptor-binding domain comprises an amino acid sequence with at least 70%, 75%, 80%, 85%, 90%, 95%, 97%, or 99% identity to any one of SEQ ID NO: 10, SEQ ID NO: 11, SEQ ID NO: 12 and SEQ ID NO: 13 and is capable of binding TrkB. Each possibility represents a separate embodiment of the invention.

The term “analog” as used herein, refers to a polypeptide that is similar, but not identical, to the polypeptide of the invention that still is capable of binding the target receptor. An analog, may have deletions or mutations that result in an amino acids sequence that is different than the amino acid sequence of the polypeptide of the invention. It should be understood, that all analogs of the polypeptide of the invention would still be capable of binding the target receptor. Further, an analog may be analogous to a fragment of the polypeptide of the invention, however, in such a case the fragment must comprise at least 50 consecutive amino acids of the polypeptide of the invention. In some embodiments, the analog or homolog is a human analog or homolog of the murine antibodies or antigen binding fragments described herein.

In some embodiments, the target receptor is TrkB and the chimeric transmembrane comprises the amino acid polypeptide sequence

(SEQ ID NO: 18)
DVVMTQLPLSLPVILGDQASISCRSSQSLIHSNGNTYLHWYLQK
PGQSPKLLIYKVSNRFSGVPDRFSGSGSGTDFTLKISRVEAEDL
GVYFCSQSTHVPFTFGSGTKLEIKRAGGSSRSSSSGGGGSGGGG
QVQLQQSGPELVKPGASVKLSCKASGYTFTSYDINWVKQRPGQG
LEWIGWIYPRDGSIKFNEKFKGKATLTVDTSSSTAYMELHSLTS
EDSAAYFCARRGRLLLYGFAYWGQGTLVTVSAXXEQKLISEEDL
ALSNSIMYFSHFVPVFLPAKPTTTPAPRPPTPAPTIASQPLSLR
PEASRPAAGGAVHTRGLDLCYLLDGILFIYGVIITALYLRAKFS
RSAETAANLQDPNQLFNELNLGRREEFDVLEKKRARDPEMGGKQ
QRRRNPQEGVFNALQKDKMAEAFSEIGTKGERRRGKGHDGLFQG
LSTATKDTFDALHMQTLAPR
or
(SEQ ID NO: 20)
DVVMTQTPLSLPVSLGDQASISCRSSQSLVHSNGNTYLHWYLQK
PGQSPNLLIYKVSNRFSGVPDRFSGSGSGTDFTLKISRVEAEDL
GVYFCSQGTHVPYTFGGGTKLEIKRAGGSSRSSSSGGGGSGGGG
QVQLQQSGAELVRPGASVTLSCKASGYTFTDYEMHWVKQTPVHG
LEWIGTIDPETAGTAYNNQKFKGKAILTAGKSSSTAYMELRSLT
SEDSAVYYCTGVTTWFAYWGQGTLVTVSAXXALSNSIMYFSHFV
PVFLPAKPTTTPAPRPPTPAPTIASQPLSLRPEASRPAAGGAVH
TRGLDLCYLLDGILFIYGVIITALYLRAKFSRSAETAANLQDPN
QLFNELNLGRREEFDVLEKKRARDPEMGGKQQRRRNPQEGVFNA
LQKDKMAEAFSEIGTKGERRRGKGHDGLFQGLSTATKDTFDALH
MQTLAPREGRGSLLTCGDVEENPGPMVSKGEELFTGVVPILVEL
DGDVNGHKFSVSGEGEGDATYGKLTLKFICTTGKLPVPWPTLVT
TLTYGVQCFSRYPDHMKQHDFFKSAMPEGYVQERTIFFKDDGNY
KTRAEVKFEGDTLVNRIELKGIDFKEDGNILGHKLEYNYNSHNV
YIMADKQKNGIKVNFKIRHNIEDGSVQLADHYQQNTPIGDGPVL
LPDNHYLSTQSALSKDPNEKRDHMVLLEFVTAAGITLGMDELYK.

In some embodiments, the target receptor is GLP1R and the target receptor-binding domain comprises a VL comprising the amino acid sequence QIVLTQSPAIMSASPGEKVTMTCSASSRVTYMHWYQQRSGTSPKRWIYDTSKL ASGVPARFSGSGSGTSYSLTISSMEAEDAATYYCQQWGNNPQYTFGGGTRLEIK R (SEQ ID NO: 14). In some embodiments, the target receptor is GLP1R and the target receptor-binding domain comprises a VH comprising the amino acid sequence QVTLKESGPGILQPSQTLSLTCSFSGFSLSTSGTGVGWIRQPSGKGLEWLSHIWW DDVKRYNPALKSRLTISRDTSYSQVFLRIASVDTADTATYYCARILDGTGPMDY WGQGTSVTVSS (SEQ ID NO: 15). In some embodiments, the target receptor is GLP1R and the target receptor-binding domain comprises SEQ ID NO: 14 and SEQ ID NO: 15.

In some embodiments, the target receptor is GLP1R and the target receptor-binding domain comprises an analog of any one of SEQ ID NO: 14 and SEQ ID NO: 15. In some embodiments, the target receptor is GLP1R and the target receptor-binding domain comprises a homolog of any one of SEQ ID NO: 14 and SEQ ID NO: 15. In some embodiments, the target receptor is GLP1R and the target receptor-binding domain comprises an amino acid sequence with at least 70%, 75%, 80%, 85%, 90%, 95%, 97%, or 99% identity to any one of SEQ ID NO: 14 and SEQ ID NO: 15 and is capable of binding GLP1R. Each possibility represents a separate embodiment of the invention.

In some embodiments, the target receptor is GLP1R and the chimeric transmembrane polypeptide comprises the amino acid sequence

(SEQ ID NO: 22)
IVLTQSPAIMSASPGEKVTMTCSASSRVTYMHWYQQRSGTSPKR
WIYDTSKLASGVPARFSGSGSGTSYSLTISSMEAEDAATYYCQQ
WGNNPQYTFGGGTRLEIKRGGGGSGGGGSGGGGSGGGGSQVTLK
ESGPGILQPSQTLSLTCSFSGFSLSTSGTGVGWIRQPSGKGLEW
LSHIWWDDVKRYNPALKSRLTISRDTSYSQVFLRIASVDTADTA
TYYCARILDGTGPMDYWGQGTSVTVSSXXEQKLISEEDLALSNS
IMYFSHFVPVFLPAKPTTTPAPRPPTPAPTIASQPLSLRPEASR
PAAGGAVHTRGLDLCYLLDGILFIYGVIITALYLRAKFSRSAET
AANLQDPNQLFNELNLGRREEFDVLEKKRARDPEMGGKQQRRRN
PQEGVFNALQKDKMAEAFSEIGTKGERRRGKGHDGLFQGLSTAT
KDTFDALHMQTLAPR.

In some embodiments, the target receptor is GHR and the target receptor-binding domain comprises a Growth Hormone (GH). In some embodiments, the GH is human GH (hGH). In some embodiments, hGh comprises the amino acid sequence EGSADYKDHDGDYKDHDIDYKDDDDKFPTIPLSRLFDNAMLRAHRLHQLAFD TYQEFEEAYIPKEQKYSFLQNPQTSLCFSESIPTPSNREETQQKSNLELLRISLLLI QSWLEPVQFLRSVFANSLVYGASDSNVYDLLKDLEEGIQTLMGRLEDGSPRTG QIFKQTYSKFDTNSHNDDALLKNYGLLYCFRKDMDKVETFLRIVQCRSVEGSC GF (SEQ ID NO: 16). In some embodiments, the GH is a fragment, homolog, analog or derivative of hGH that is capable of binding GHR. In some embodiments, the GH comprises an amino acid sequence with at least 70%, 75%, 80%, 85%, 90%, 95%, 97%, or 99% identity to SEQ ID NO: 16.

The term “derivative” as used herein, refers to any polypeptide that is based off the polypeptide of the invention and is still capable of binding the target receptor. A derivative is not merely a fragment of the polypeptide, nor does it have amino acids replaced or removed (an analog), rather it may have additional modification made to the polypeptide, such as a post-translational modification. Further, a derivative may be a derivative of a fragment of the polypeptide of the invention, however, in such a case the fragment must comprise at least 50 consecutive amino acids of the polypeptide of the invention. In some embodiments, the chimeric transmembrane polypeptides of the invention are derivatives of the polypeptides described herein. In some embodiments, the derivatives comprise glycosylation of the polypeptide. One skilled in the art will appreciate that glycosylation of the polypeptide may be necessary for robust surface expression.

In some embodiments, the target receptor is GHR and the chimeric transmembrane polypeptide comprises the amino acid sequence

(SEQ ID NO: 24)
EGSADYKDHDGDYKDHDIDYKDDDDKFPTIPLSRLFDNAMLRAH
RLHQLAFDTYQEFEEAYIPKEQKYSFLQNPQTSLCFSESIPTPS
NREETQQKSNLELLRISLLLIQSWLEPVQFLRSVFANSLVYGAS
DSNVYDLLKDLEEGIQTLMGRLEDGSPRTGQIFKQTYSKFDTNS
HNDDALLKNYGLLYCFRKDMDKVETFLRIVQCRSVEGSCGFXXE
QKLISEEDLALSNSIMYFSHFVPVFLPAKPTTTPAPRPPTPAPT
IASQPLSLRPEASRPAAGGAVHTRGLDLCYLLDGILFIYGVIIT
ALYLRAKFSRSAETAANLQDPNQLFNELNLGRREEFDVLEKKRA
RDPEMGGKQQRRRNPQEGVFNALQKDKMAEAFSEIGTKGERRRG
KGHDGLFQGLSTATKDTFDALHMQTLAPR.

In some embodiments, the target receptor is PD-1 and the target receptor-binding domain comprises a VL comprising the amino acid sequence EIVLTQSPATLSLSPGERATLSCRASKGVSTSGYSYLHWYQQKPGQAPRLLIYLA SYLESGVPARFSGSGSGTDFTLTISSLEPEDFAVYYCQHSRDLPLTFGGGTKVEI KRTVAAPSVFIFPPSDEQLKSGTASVVCLLNNFYPREAKVQWKVDNALQSGNS QESVTEQDSKDSTYSLSSTLTLSKADYEKHKVYACEVTHQGLSSPVTKSFNRGE C (SEQ ID NO: 25). In some embodiments, the target receptor is PD-1 and the target receptor-binding domain comprises a VH comprising the amino acid sequence QVQLVQSGVEVKKPGASVKVSCKASGYTFTNYYMYWVRQAPGQGLEWMGGI NPSNGGTNFNEKFKNRVTLTTDSSTTTAYMELKSLQFDDTAVYYCARRDYRFD MGFDYWGQGTTVTVSSASTKGPSVFPLAPCSRSTSESTAALGCLVKDYFPEPVT VSWNSGALTSGVHTFPAVLQSSGLYSLSSVVTVPSSSLGTKTYTCNVDHKPSNT KVDKRVESKYGPPCPPCPAPEFLGGPSVFLFPPKPKDTLMISRTPEVTCVVVDVS QEDPEVQFNWYVDGVEVHNAKTKPREEQFNSTYRVVSVLTVLHQDWLNGKE YKCKVSNKGLPSSIEKTISKAKGQPREPQVYTLPPSQEEMTKNQVSLTCLVKGF YPSDIA VEWESNGQPENNYKTTPPVLDSDGSFFLYSRLTVDKSRWQEGNVFSCS VMHEALHNHYTQKSLSLSLGK (SEQ ID NO: 26). In some embodiments, the target receptor is PD-1 and the target receptor-binding domain comprises SEQ ID NO:25 and SEQ ID NO: 26.

In some embodiments, the target receptor is PD-1 and the target receptor-binding domain comprises an analog of any one of SEQ ID NO: 25 and SEQ ID NO: 26. In some embodiments, the target receptor is PD-1 and the target receptor-binding domain comprises a homolog of any one of SEQ ID NO: 25 and SEQ ID NO: 26. In some embodiments, the target receptor is PD-1 and the target receptor-binding domain comprises an amino acid sequence with at least 70%, 75%, 80%, 85%, 90%, 95%, 97%, or 99% identity to any one of SEQ ID NO: 25 and SEQ ID NO: 26 and is capable of binding PD-1. Each possibility represents a separate embodiment of the invention.

In some embodiments, the target receptor is PD-1 and the chimeric transmembrane polypeptide comprises the amino acid sequence

(SEQ ID NO: 28)
QVQLVQSGVEVKKPGASVKVSCKASGYTFTNYYMYWVRQAPGQG
LEWMGGINPSNGGTNFNEKFKNRVTLTTDSSTTTAYMELKSLQF
DDTAVYYCARRDYRFDMGFDYWGQGTTVTVSSASTKGPSVFPLA
PCSRSTSESTAALGCLVKDYFPEPVTVSWNSGALTSGVHTFPAV
LQSSGLYSLSSVVTVPSSSLGTKTYTCNVDHKPSNTKVDKRVES
KYGPPCPPCPAPEFLGGPSVFLFPPKPKDTLMISRTPEVTCVVV
DVSQEDPEVQFNWYVDGVEVHNAKTKPREEQFNSTYRVVSVLTV
LHQDWLNGKEYKCKVSNKGLPSSIEKTISKAKGQPREPQVYTLP
PSQEEMTKNQVSLTCLVKGFYPSDIAVEWESNGQPENNYKTTPP
VLDSDGSFFLYSRLTVDKSRWQEGNVFSCSVMHEALHNHYTQKS
LSLSLGKGGSSRSSSSGGGGSGGGGEIVLTQSPATLSLSPGERA
TLSCRASKGVSTSGYSYLHWYQQKPGQAPRLLIYLASYLESGVP
ARFSGSGSGTDFTLTISSLEPEDFAVYYCQHSRDLPLTFGGGTK
VEIKRTVAAPSVFIFPPSDEQLKSGTASVVCLLNNFYPREAKVQ
WKVDNALQSGNSQESVTEQDSKDSTYSLSSTLTLSKADYEKHKV
YACEVTHQGLSSPVTKSFNRGECXXEQKLISEEDLALSNSIMYF
SHFVPVFLPAKPTTTPAPRPPTPAPTIASQPLSLRPEASRPAAG
GAVHTRGLDLCYLLDGILFIYGVIITALYLRAKFSRSAETAANL
QDPNQLFNELNLGRREEFDVLEKKRARDPEMGGKQQRRRNPQEG
VFNALQKDKMAEAFSEIGTKGERRRGKGHDGLFQGLSTATKDTF
DALHMQTLAPR.

The XX in all of the sequence may be any two amino acids, or no amino acids, as these amino acids are a result of a restriction enzyme site in the coding sequence of the chimeric protein and have no functional role. In some embodiments, the XX is valine-aspartic acid.

In some embodiments, the chimeric transmembrane polypeptides of the invention further comprise a leader peptide at their N-terminus. In some embodiments, the leader peptide comprises or consists of the sequence MDMRVPAQLLGLLLLWLSGARCQ (SEQ ID NO: 2).

In some embodiments, the chimeric transmembrane polypeptide has at least 70%, 75%, 80%, 85%, 90%, 95%, 97%, or 99% homology or identity to an amino acid sequence of any one of SEQ ID NOs: 18, 20, 22, 24 or 28 and is not capable of transducing an immune cell activating signal. Each possibility represents a separate embodiment of the invention. In some embodiments, the chimeric transmembrane polypeptide is a derivative or analog of any one of SEQ ID NOs: 18, 20, 22, 24 or 28. Each possibility represents a separate embodiment of the invention.

### Pharmaceutical Compositions

By another aspect, there is provided a pharmaceutical composition comprising any of the modified cells of the invention and a pharmaceutically acceptable carrier, excipient, or adjuvant. In some embodiments, the pharmaceutical composition comprises a therapeutically effective amount of the modified cells of the invention.

As used herein, the term “carrier,” “adjuvant” or “excipient” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

The term “therapeutically effective amount” refers to a number of cells effective to treat a disease or disorder in a mammal. The term “a therapeutically effective amount” refers to an amount effective, at dosages and for periods of time necessary, to achieve the desired therapeutic or prophylactic result. The exact dosage form and regimen would be determined by the physician according to the patient's condition.

In some embodiments, the pharmaceutical composition comprises at least 1 million, 2 million, 3 million, 5 million, 10 million, 50 million or 100 million immune cells. Each possibility represents a separate embodiment of the invention.

### Methods of Treatment

In some embodiments, the providing comprises extracting a primary cell from the subject. In some embodiments, extracting a primary cell from a subject comprises drawing a blood or serum sample. In some embodiments, extracting a primary cell from a subject comprises drawing a lymph sample. In some embodiments, a primary cell is isolation using a kit. Such kits are common in the art and include, but are not limited to, Miltenyi cell isolation and cell separation kits, CD4+ magnetic bead kits, CD8+ magnetic bead kits, and the like. In some embodiments, the primary cell is isolated using an antibody conjugated column. In some embodiments, a primary cell is isolated using FACS sorting. Any suitable FACS antibody that identifies are target cell may be used for the cell sorting.

Expression of heterologous proteins in a target cell is well known in the art. Any method whereby the chimeric transmembrane proteins of the invention are expressed in the cell may be used to perform the methods of the invention.

The term “expression” as used herein refers to the biosynthesis of a protein including translation of said gene product. Thus, expression of a protein may refer to transcription of the nucleic acid fragment (e.g., transcription resulting in mRNA or other functional RNA) and/or translation of RNA into a precursor or mature protein (polypeptide). In some embodiments, expression is expression on the cell surface. In some embodiments, expression is expression of a precursor protein comprising a leader peptide, cleavage of that peptide and surface expression of the mature protein.

Expressing of a heterologous transcript within a cell is well known to one skilled in the art. It can be carried out by, among many methods, transfection, viral infection, or direct alteration of the cell's genome. In some embodiments, the heterologous transcript is in an expression vector such as plasmid or viral vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the heterologous transcript is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

It will be appreciated that other than containing the necessary elements for the transcription and translation of the inserted coding sequence (encoding the chimeric transmembrane polypeptide), the expression construct of the present invention can also include sequences engineered to optimize stability, production, purification, yield or activity of the expressed polypeptide.

By another aspect, there is provided an artificial expression vector that encodes a chimeric transmembrane protein of the invention.

As used herein, the terms “administering,” “administration,” and like terms refer to any method which, in sound medical practice, delivers a composition containing an active agent to a subject in such a manner as to provide a therapeutic effect. Other suitable routes of administration can include parenteral, subcutaneous, intravenous, intramuscular, intracranial, intracerebroventricular, intrathecal, or intraperitoneal. It will be understood by one skilled in the art that due to the homing capabilities of immune cells systemic administration is sufficient for treatment of even local aliments. In some embodiments, administration is performed directly to the site of disease. Non-limiting examples of such are intracranial administration for a brain disease, topical administration for a skin disease, and intramuscular administration for a muscle disease.

The dosage administered will be dependent upon the age, health, and weight of the recipient, kind of concurrent treatment, if any, frequency of treatment, and the nature of the effect desired.

In some embodiments, the modified cell is administered to the subject. In some embodiments, a therapeutically effective amount of modified cells is administered to the subject. In some embodiments, a pharmaceutical composition comprising a therapeutically effective amount of modified cells is administered to the subject.

In some embodiments, the methods of the invention may be used to treat any disease, disorder or condition for which a target molecule is known to be associated and wherein activation or inhibition of that molecule is known to have a positive effect on the disease, disorder or condition. In some embodiments, a positive effect is any improvement in a symptom of that disease, disorder or condition. In some embodiments, a positive effect is any reduction in the severity, or longevity of a symptom. In some embodiments, a positive effect is any improvement in the quality of life of the subject.

In some embodiments, the target receptor is TrkB and the disease or disorder is a neurological disease or disorder. In some embodiments, the neurological disease or disorder is selected from: Alzheimer's disease, depression, memory loss, amyotrophic lateral sclerosis (ALS), epilepsy and brain cancer. In some embodiments, the neurological disease or disorder is a Brain-Derived Neurotrophic Factor (BDNF)-associated disease or disorder. In some embodiments, the target receptor-binding domain comprises an anti-TrkB antigen binding domain. In some embodiments, the TrkB antigen binding domain is from a commercially available anti-TrkB antibody.

In some embodiments, the target receptor is GLP1R and the disease or disorder is a metabolic or cardiovascular disease or disorder. In some embodiments, a metabolic disease or disorder is selected from: diabetes, obesity, glycogen storage disease, Parkinson's disease and mitochondrial myopathy. In some embodiments, a metabolic disease or disorder is a mitochondrial disease or disorder. In some embodiments, the metabolic disease is a disease of glucose homeostasis. In some embodiments, the cardiovascular disease or disorder is selected from: stroke, myocardial infarction, cardiac ischemia, and coronary artery disease. In some embodiments, the target receptor-binding domain comprises an anti-GLP1R antigen binding domain. In some embodiments, the GLP1R antigen binding domain is from a commercially available anti-GLP1R antibody.

In some embodiments, the target receptor is GHR and the disease or disorder is a growth disease or disorder. In some embodiments, the growth disease or disorder is selected from: acromegaly, growth hormone deficiency, cancer, Turner syndrome, and Prader-Willi syndrome. In some embodiments, the growth disease or disorder is any disease or disorder which can be treated with administration of hGH. In some embodiments, the disease which can be treated with hGH is a muscle disease. In some embodiments, the muscle disease is selected from a muscle wasting disease and a muscular dystrophy. In some embodiments, the muscle wasting disease is selected from multiple sclerosis, cachexia and sarcopenia. In some embodiments, the muscular dystrophy is selected from Deschene's muscular dystrophy, Becker muscular dystrophy, myotonic dystrophy, and facioscapulohumeral muscular dystrophy. In some embodiments, the target receptor-binding domain comprises a GH. In some embodiments, the GH is hGH.

In some embodiments, the target receptor is PD-1 and the disease or disorder is an immune disease or disorder or cancer. In some embodiments, the immune disease or disorder is selected from: lupus, rheumatoid arthritis, psoriasis, Graves' disease, immune-mediated inflammation, and celiac disease. In some embodiments, the disease or disorder is cancer and the target receptor-binding domain comprises a PD-1 antagonist. In some embodiments, the target receptor-binding domain comprises an anti-PD-1 antigen binding domain. In some embodiments, the PD-1 antigen binding domain is from a commercially available anti-PD-1 antibody.

Non-limiting examples for GLP1-R agonist antibodies that can be used as a source for GLP1-R variable heavy and light chains are described in US patent application numbers: 2014/0911910, 2014/0775074, 2013/0650469, 20150965841, 2009/0392244, 2007/0374569 and US patents numbers: 9,358,287; 8,501,693 and 9,328,154 incorporated herein by reference.

Non-limiting examples for TrkB agonist antibodies that can be used as a source for TrkB variable heavy and light chains are described in US patent application numbers: 2007/0516187, 2008/0682505, 2010/0697983 and U.S. Pat. Nos. 7,459,156; 7,750,122; 8,642,035; 9,028,820 incorporated herein by reference.

Non-limiting examples for PD-1 or PD-L1 agonist antibodies that can be used as a source for variable heavy and light chains are described in U.S. Pat. Nos. 7,427,665; 7,722,868; 7,595,048; 7,488,802; 8,008,449; 7,943,743; 9,181,342 and 8,617,546 incorporated herein by reference.

Non-limiting examples for CTLA4 agonist antibodies that can be used as a source for CTLA4 variable heavy and light chains are described in US patent numbers: 7,592,007; 7,109,003; 7,034,121; 7,605,238; 7,452,535 incorporated herein by reference.

In some embodiments, the methods of the invention further comprise partially activating the modified cell. In some embodiments, partial activation comprises homing ability and/or the ability to synapse with the target cell. In some embodiments, partial activation does not comprise effector function activation and/or activation of cytotoxicity.

In some embodiments, the methods of the invention further comprise determining modulation of signaling in the target cell. In some embodiments, the methods of the invention further comprise determining modulation of the target receptor. In some embodiments, the methods of the invention further comprise determining the modified cell is not cytotoxic. In some embodiments, the methods of the invention further comprise determining the modified immune cell is not activated.

In some embodiments, the determining comprises determining phosphorylation of at least one signaling protein. In some embodiments, the determining comprises determining phosphorylation of a residue within a signaling domain of the target receptor. In some embodiments, the determining comprises determining phosphorylation of a residue within a signaling domain of a protein of a signaling cascade. In some embodiments, the phosphorylated residue is a tyrosine residue. In some embodiments, the determining comprises determining upregulation of a level of a downstream target of the target receptor. In some embodiments, the determining comprises determining down-regulation of a level of a downstream target of the target receptor.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells-A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

### Cloning of Chimeric Transmembrane Proteins and Retroviral Transduction of BW5147 Cells

Chimeric transmembrane (TM) protein sequences include leader peptide-VL-linker-VH-hinge-TM cytoplasmic part-GFP (TrkB-GFP-WT) and VL-linker-VH-hinge-mutated TM cytoplasmic part-GFP (TrkB-GFP-Mut) and VL-linker-VH-Myc-hinge-TM cytoplasmic part (TrkB-Myc-WT) and VL-linker-VH-Myc-hinge-mutated TM cytoplasmic part (TrkB-Myc-Mut) were synthesized for anti-TrkB. Two chimeric transmembrane protein sequences include leader peptide-VL-linker-VH-MYC+hinge-TM-cytoplasmic part were synthesized for GLP1R and PD-1.

The WT murine zeta TM-cytoplasmic sequence used is LCYLLDGILFIYGVIITALYLRAKFSRSAETAANLQDPNQLYNELNLGRREEYDV LEKKRARDPEMGGKQQRRRNPQEGVYNALQKDKMAEAYSEIGTKGERRRGK GHDGLYQGLSTATKDTYDALHMQTLAPR (SEQ ID NO: 8). The mutant zeta TM-cytoplasmic sequence used is

(SEQ ID NO: 9)
LCYLLDGILFIYGVIITALYLRAKFSRSAETAANLQDPNQLFNELNLG
RREEFDVLEKKRARDPEMGGKQQRRRNPQEGVFNALQKDKMAEAFSEI
GTKGERRRGKGHDGLFQGLSTATKDTFDALHMQTLAPR.

The 8 sequences generated were the following, with an N-terminal leader peptide of MDMRVPAQLLGLLLLWLSGARCQ (SEQ ID NO: 2):

TrkB-Myc-WT:
(SEQ ID NO: 17)
DVVMTQLPLSLPVILGDQASISCRSSQSLIHSNGNTYLHWYLQKPGQS
PKLLIYKVSNRFSGVPDRFSGSGSGTDFTLKISRVEAEDLGVYFCSQS
THVPFTFGSGTKLEIKRAGGSSRSSSSGGGGSGGGGQVQLQQSGPELV
KPGASVKLSCKASGYTFTSYDINWVKQRPGQGLEWIGWIYPRDGSIKF
NEKFKGKATLTVDTSSSTAYMELHSLTSEDSAAYFCARRGRLLLYGFA
YWGQGTLVTVSAXXEQKLISEEDLALSNSIMYFSHFVPVFLPAKPTTT
PAPRPPTPAPTIASQPLSLRPEASRPAAGGAVHTRGLDLCYLLDGILF
IYGVIITALYLRAKFSRSAETAANLQDPNQLYNELNLGRREEYDVLEK
KRARDPEMGGKQQRRRNPQEGVYNALQKDKMAEAYSEIGTKGERRRGK
GHDGLYQGLSTATKDTYDALHMQTLAPR.
TrkB-Myc-Mut:
(SEQ ID NO: 18)
DVVMTQLPLSLPVILGDQASISCRSSQSLIHSNGNTYLHWYLQKPGQS
PKLLIYKVSNRFSGVPDRFSGSGSGTDFTLKISRVEAEDLGVYFCSQS
THVPFTFGSGTKLEIKRAGGSSRSSSSGGGGSGGGGQVQLQQSGPELV
KPGASVKLSCKASGYTFTSYDINWVKQRPGQGLEWIGWIYPRDGSIKF
NEKFKGKATLTVDTSSSTAYMELHSLTSEDSAAYFCARRGRLLLYGFA
YWGQGTLVTVSAXXEQKLISEEDLALSNSIMYFSHFVPVFLPAKPTTT
PAPRPPTPAPTIASQPLSLRPEASRPAAGGAVHTRGLDLCYLLDGILF
IYGVIITALYLRAKFSRSAETAANLQDPNQLFNELNLGRREEFDVLEK
KRARDPEMGGKQQRRRNPQEGVFNALQKDKMAEAFSEIGTKGERRRGK
GHDGLFQGLSTATKDTFDALHMQTLAPR.
TrkB-GFP-WT:
(SEQ ID NO: 19)
DVVMTQTPLSLPVSLGDQASISCRSSQSLVHSNGNTYLHWYLQKPGQS
PNLLIYKVSNRFSGVPDRFSGSGSGTDFTLKISRVEAEDLGVYFCSQG
THVPYTFGGGTKLEIKRAGGSSRSSSSGGGGSGGGGQVQLQQSGAELV
RPGASVTLSCKASGYTFTDYEMHWVKQTPVHGLEWIGTIDPETAGTAY
NNQKFKGKAILTAGKSSSTAYMELRSLTSEDSAVYYCTGVTTWFAYWG
QGTLVTVSAXXALSNSIMYFSHFVPVFLPAKPTTTPAPRPPTPAPTIA
SQPLSLRPEASRPAAGGAVHTRGLDLCYLLDGILFIYGVIITALYLRA
KFSRSAETAANLQDPNQLYNELNLGRREEYDVLEKKRARDPEMGGKQQ
RRRNPQEGVYNALQKDKMAEAYSEIGTKGERRRGKGHDGLYQGLSTAT
KDTYDALHMQTLAPREGRGSLLTCGDVEENPGPMVSKGEELFTGVVPI
LVELDGDVNGHKFSVSGEGEGDATYGKLTLKFICTTGKLPVPWPTLVT
TLTYGVQCFSRYPDHMKQHDFFKSAMPEGYVQERTIFFKDDGNYKTRA
EVKFEGDTLVNRIELKGIDFKEDGNILGHKLEYNYNSHNVYIMADKQK
NGIKVNFKIRHNIEDGSVQLADHYQQNTPIGDGPVLLPDNHYLSTQSA
LSKDPNEKRDHMVLLEFVTAAGITLGMDELYK.
TrkB-GFP-Mut:
(SEQ ID NO: 20)
DVVMTQTPLSLPVSLGDQASISCRSSQSLVHSNGNTYLHWYLQKPGQS
PNLLIYKVSNRFSGVPDRFSGSGSGTDFTLKISRVEAEDLGVYFCSQG
THVPYTFGGGTKLEIKRAGGSSRSSSSGGGGSGGGGQVQLQQSGAELV
RPGASVTLSCKASGYTFTDYEMHWVKQTPVHGLEWIGTIDPETAGTAY
NNQKFKGKAILTAGKSSSTAYMELRSLTSEDSAVYYCTGVTTWFAYWG
QGTLVTVSAXXALSNSIMYFSHFVPVFLPAKPTTTPAPRPPTPAPTIA
SQPLSLRPEASRPAAGGAVHTRGLDLCYLLDGILFIYGVIITALYLRA
KFSRSAETAANLQDPNQLFNELNLGRREEFDVLEKKRARDPEMGGKQQ
RRRNPQEGVFNALQKDKMAEAFSEIGTKGERRRGKGHDGLFQGLSTAT
KDTFDALHMQTLAPREGRGSLLTCGDVEENPGPMVSKGEELFTGVVPI
LVELDGDVNGHKFSVSGEGEGDATYGKLTLKFICTTGKLPVPWPTLVT
TLTYGVQCFSRYPDHMKQHDFFKSAMPEGYVQERTIFFKDDGNYKTRA
EVKFEGDTLVNRIELKGIDFKEDGNILGHKLEYNYNSHNVYIMADKQK
NGIKVNFKIRHNIEDGSVQLADHYQQNTPIGDGPVLLPDNHYLSTQSA
LSKDPNEKRDHMVLLEFVTAAGITLGMDELYK.
GLP1R-Myc-WT:
(SEQ ID NO: 21)
IVLTQSPAIMSASPGEKVTMTCSASSRVTYMHWYQQRSGTSPKRWIYD
TSKLASGVPARFSGSGSGTSYSLTISSMEAEDAATYYCQQWGNNPQYT
FGGGTRLEIKRGGGGSGGGGSGGGGSGGGGSQVTLKESGPGILQPSQT
LSLTCSFSGFSLSTSGTGVGWIRQPSGKGLEWLSHIWWDDVKRYNPAL
KSRLTISRDTSYSQVFLRIASVDTADTATYYCARILDGTGPMDYWGQG
TSVTVSSXXEQKLISEEDLALSNSIMYFSHFVPVFLPAKPTTTPAPRP
PTPAPTIASQPLSLRPEASRPAAGGAVHTRGLDLCYLLDGILFIYGVI
ITALYLRAKFSRSAETAANLQDPNQLYNELNLGRREEYDVLEKKRARD
PEMGGKQQRRRNPQEGVYNALQKDKMAEAYSEIGTKGERRRGKGHDGL
YQGLSTATKDTYDALHMQTLAPR.
GLP1R-Myc-Mut:
(SEQ ID NO: 22)
IVLTQSPAIMSASPGEKVTMTCSASSRVTYMHWYQQRSGTSPKRWIYD
TSKLASGVPARFSGSGSGTSYSLTISSMEAEDAATYYCQQWGNNPQYT
FGGGTRLEIKRGGGGSGGGGSGGGGSGGGGSQVTLKESGPGILQPSQT
LSLTCSFSGFSLSTSGTGVGWIRQPSGKGLEWLSHIWWDDVKRYNPAL
KSRLTISRDTSYSQVFLRIASVDTADTATYYCARILDGTGPMDYWGQG
TSVTVSSXXEQKLISEEDLALSNSIMYFSHFVPVFLPAKPTTTPAPRP
PTPAPTIASQPLSLRPEASRPAAGGAVHTRGLDLCYLLDGILFIYGVI
ITALYLRAKFSRSAETAANLQDPNQLFNELNLGRREEFDVLEKKRARD
PEMGGKQQRRRNPQEGVFNALQKDKMAEAFSEIGTKGERRRGKGHDGL
FQGLSTATKDTFDALHMQTLAPR.
PD-1-Myc-WT:
(SEQ ID NO: 27)
QVQLVQSGVEVKKPGASVKVSCKASGYTFTNYYMYWVRQAPGQGLEWM
GGINPSNGGTNFNEKFKNRVTLTTDSSTTTAYMELKSLQFDDTAVYYC
ARRDYRFDMGFDYWGQGTTVTVSSASTKGPSVFPLAPCSRSTSESTAA
LGCLVKDYFPEPVTVSWNSGALTSGVHTFPAVLQSSGLYSLSSVVTVP
SSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEAL
HNHYTQKSLSLSLGKGGSSRSSSSGGGGSGGGGEIVLTQSPATLSLSP
GERATLSCRASKGVSTSGYSYLHWYQQKPGQAPRLLIYLASYLESGVP
ARFSGSGSGTDFTLTISSLEPEDFAVYYCQHSRDLPLTFGGGTKVEIK
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSENRGECXXEQKLISEEDLALSNSIMYFSHEVPVFLPAKPTTTP
APRPPTPAPTIASQPLSLRPEASRPAAGGAVHTRGLDLCYLLDGILFI
YGVIITALYLRAKFSRSAETAANLQDPNQLYNELNLGRREEYDVLEKK
RARDPEMGGKQQRRRNPQEGVYNALQKDKMAEAYSEIGTKGERRRGKG
HDGLYQGLSTATKDTYDALHMQTLAPR
PD-1-Myc-Mut:
(SEQ ID NO: 28)
QVQLVQSGVEVKKPGASVKVSCKASGYTFTNYYMYWVRQAPGQGLEWM
GGINPSNGGTNFNEKFKNRVTLTTDSSTTTAYMELKSLQFDDTAVYYC
ARRDYRFDMGFDYWGQGTTVTVSSASTKGPSVFPLAPCSRSTSESTAA
LGCLVKDYFPEPVTVSWNSGALTSGVHTFPAVLQSSGLYSLSSVVTVP
SSSLGTKTYTCNVDHKPSNTKVDKRVESKYGPPCPPCPAPEFLGGPSV
FLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESN
GQPENNYKTTPPVLDSDGSFFLYSRLTVDKSRWQEGNVFSCSVMHEAL
HNHYTQKSLSLSLGKGGSSRSSSSGGGGSGGGGEIVLTQSPATLSLSP
GERATLSCRASKGVSTSGYSYLHWYQQKPGQAPRLLIYLASYLESGVP
ARFSGSGSGTDFTLTISSLEPEDFAVYYCQHSRDLPLTFGGGTKVEIK
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSENRGECXXEQKLISEEDLALSNSIMYFSHEVPVFLPAKPTTTP
APRPPTPAPTIASQPLSLRPEASRPAAGGAVHTRGLDLCYLLDGILFI
YGVIITALYLRAKFSRSAETAANLQDPNQLFNELNLGRREEFDVLEKK
RARDPEMGGKQQRRRNPQEGVFNALQKDKMAEAFSEIGTKGERRRGKG
HDGLFQGLSTATKDTFDALHMQTLAPR.

Two more sequences were generated as above, with hGH amino acid sequence used in place of the the VL and VH. The endogenous hGH leader peptide MATGSRTSLLLAFGLLCLPWLQ (SEQ ID NO: 34) was used.

GHR-Myc-WT:
(SEQ ID NO: 23)
EGSADYKDHDGDYKDHDIDYKDDDDKFPTIPLSRLFDNAMLRAHRLHQ
LAFDTYQEFEEAYIPKEQKYSFLQNPQTSLCFSESIPTPSNREETQQK
SNLELLRISLLLIQSWLEPVQFLRSVFANSLVYGASDSNVYDLLKDLE
EGIQTLMGRLEDGSPRTGQIFKQTYSKFDTNSHNDDALLKNYGLLYCF
RKDMDKVETFLRIVQCRSVEGSCGFXXEQKLISEEDLALSNSIMYFSH
FVPVFLPAKPTTTPAPRPPTPAPTIASQPLSLRPEASRPAAGGAVHTR
GLDLCYLLDGILFIYGVIITALYLRAKFSRSAETAANLQDPNQLYNEL
NLGRREEYDVLEKKRARDPEMGGKQQRRRNPQEGVYNALQKDKMAEAY
SEIGTKGERRRGKGHDGLYQGLSTATKDTYDALHMQTLAPR.
(SEQ ID NO: 24)
EGSADYKDHDGDYKDHDIDYKDDDDKFPTIPLSRLFDNAMLRAHRLHQ
LAFDTYQEFEEAYIPKEQKYSFLQNPQTSLCFSESIPTPSNREETQQK
SNLELLRISLLLIQSWLEPVQFLRSVFANSLVYGASDSNVYDLLKDLE
EGIQTLMGRLEDGSPRTGQIFKQTYSKFDTNSHNDDALKNYGLLYCFR
KDMDKVETFLRIVQCRSVEGSCGFXXEQKLISEEDLALSNSIMYFSHF
VPVFLPAKPTTTPAPRPPTPAPTIASQPLSLRPEASRPAAGGAVHTRG
LDLCYLLDGILFIYGVIITALYLRAKFSRSAETAANLQDPNQLFNELN
LGRREEFDVLEKKRARDPEMGGKQQRRRNPQEGVFNALQKDKMAEAFS
EIGTKGERRRGKGHDGLFQGLSTATKDTFDALHMQTLAPR

In all of the above described sequences the XX maybe any amino acids or no amino acids. The XX result from a restriction enzyme site in the nucleic acid sequence that codes for the protein. In the actual proteins used the XX was valine-aspartic acid.

All 10 sequences were cloned into the retroviral vector pMP71-G-PRE. Plasmids were amplified using DH5alpha (Invitrogen) and purified with a Maxiprep Plasmid DNA Kit (Invirtogen). The packaging cell line Platinum-E (Cellbiolabs) was transfected in a 10 cm plate with 20 μg of plasmid DNA and 60 μL of PolyJet™M (SignaGen). After 16 hours, the medium was replaced to 10 ml of RPMI complete media. After 24 and 48 hours, the retrovirus supernatant was collected and filtered through a 0.45-μm filter. BW5147 cells (1×10⁶ cells/mL) were plated with virus supernatant and were spinoculated on RetroNectin-coated plates (12.5 μg/mL; TaKaRa, Clonthech) at 1500 g for 90 minutes at 32° C. with 4 μg/mL protamine sulfate (Sigma-Aldrich).

### Cloning of TrkB-T2A and Retroviral Infection of RAW5147, 3T3 and HEK293 Cells.

Mouse TrkB cDNA (Sino Biological) was cloned in the pMP71-PRE expression vector with T2A-GFP. The packaging cell line Platinum-E (Cellbiolabs) was transfected in a 10 cm plate with 20 μg of plasmid DNA and 60 μL of PolyJetTM (SignaGen). After 16 hours, the medium was replaced to 10 ml of DMEM complete media. After 24 and 48 hours, the retrovirus supernatant was collected and filtered through a 0.45-μm filter. Briefly, human embryonic kidney cells (HEK293T) cells, RAW 5147 and 3T3 cells were seeded in 24 well plates (NUNC). Next day cells were incubated with viral supernatant with 4 μg/mL protamine sulfate (Sigma-Aldrich). Transduced T cells were stained and analyzed 48-72 hours after sorting (FACS Aria cell sorter (BD biosciences, San Jose, CA, USA)

### In Vitro Study of the Interaction Between TrkB Rreceptor and Chimeric-TrkB Antibody

In cell-based binding assays, Raw264.7 cells expressing TrkB receptor were seeded on 12 well plates (Nunc) in DMEM based media (10% FBS, PSN). After reaching 85-95% confluency, cells were twice washed and DMEM meida with 0.2% FBS was added for 4 hours. Then cells were washed and incubated during 1 h with DMEM without FBS before addition of neurotrophins or BW5147 cells. After that TrkB-expressing Raw 264.7 cells were incubated with BDNF (50 ng/ml) and BW5147 cells with chimeric receptors for 30 min and 1 h., Co-cultured cells were harvested and processed for Western blot analysis.

### Western Blot Analysis

Cells were lysed in RIPA buffer (50 mM Tris-Cl, pH 8.0, 150 mM NaCl, 1% Triton, 0.5% sodium dodecyl sulphate) containing protease inhibitors (Sigma) and phosphatase inhibitors (Sigma). 30-50 μg of lysate was separated on 12% Tris-Glycine SDS-PAGE gels and then transferred to PVDF or nitrocellulose membranes. Membranes were incubated with antibodies against pTrkB (Tyr706/707), total TrkB (TrkB (80E3) from Cell Signaling and actin. WesternBright Quantum (Advansta) was used for visualization of the signal. The images were captured using a bioimaging analyzer (Fusion-FX; Vilber, France) and analyzed using ImageJ program. Quantification of the Western blots of phosphorylated TrkB. The integrated density value of the bands in Western blots was determined using densitometry (ImageJ), and data was normalized to actin and to total TrkB.

### Cytokine ELISA

Raw264.7 cells expressing TrkB receptor and Raw264.7 cells were seeded on 96 well plates (Nunc) in DMEM based media (10% FBS, PSN). After reaching 85-95% confluency, cells were twice washed and 100 μl of DMEM meida with 0.2% FBS was added for 4 hours. After 4 hours BW5147 cells in were added to the wells in 100 μl of DMEM media without FBS for ON. Supernatants were collected after 16 hours and analyzed for IL-2 with a sandwich ELISA (BioLegend), according to the manufacturer's instructions. Samples were analyzed with triplicates.

### Statistical Analyses

All statistical analyses were performed with GraphPad Prism version 5.02 for Windows (GraphPad Software, San Diego, CA). All variables are expressed as means 6 SEM or SD, as indicated in figure legends. The p-values were calculated with one-way Anova test.

### Example 1. BW Cells Expressing αTrkB-Myc or αTrkB-GFP Chimeric Receptors Cells Induce the Phosphorylation of TrkB in Target RAW-TrkB Cells

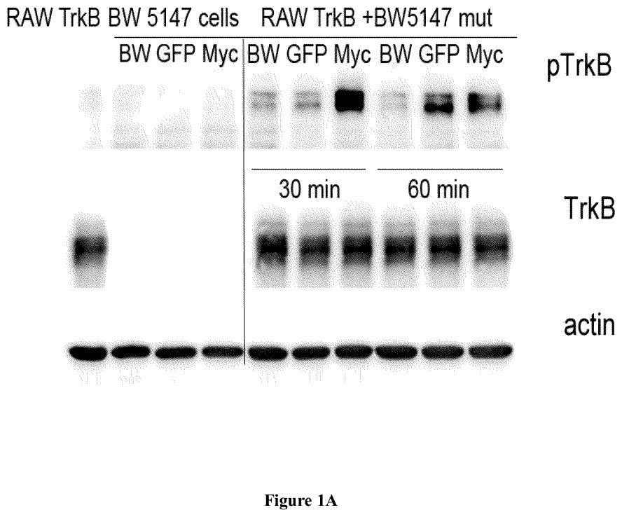

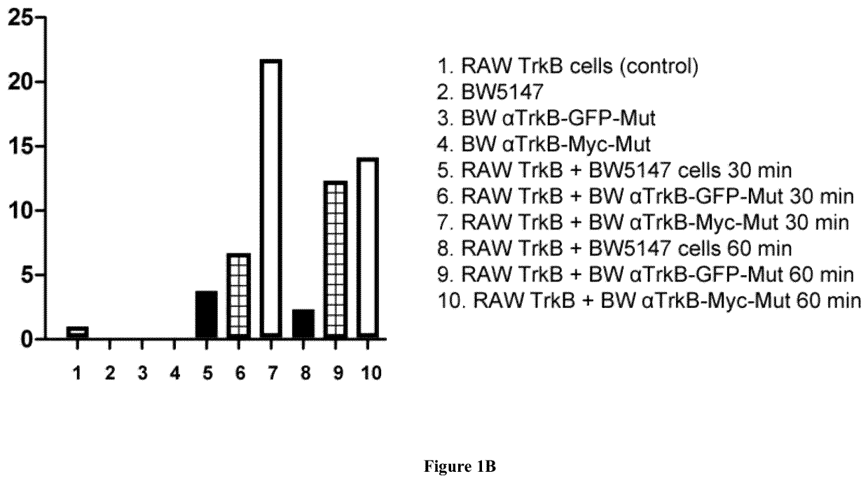

To test the ability of TRAMMICS expressing cells to induce phosphorylation of the tyrosine receptor kinase B (TrkB) receptor on target cells, the following experiment was performed. RAW 264.7 cells stably expressing TrkB were generated. These RAW-TrkB cells were incubated with untransformed BW5147, BW-αTrkB-GFP with mutant (Mut) zeta chain (SEQ ID NO: 20), and BW-αTrkB-Myc-Mut (SEQ ID NO: 18), for 30 and 60 min. The results of this experiment are presented in FIGS. 1A-B. The protein band at 145 kDa, corresponding to the phosphorylation of TrkB at tyrosine 706, can be seen in FIG. 1A and is quantified in FIG. 1B. Coculture with BW-αTrkB-GFP-Mut (FIG. 1B, #6) and to a greater extent BW-αTrkB-Myc-Mut (FIG. 1B, #7) cells induced an increase in the level of phosphorylated TrkB (pTrkB) in RAW-TrkB cells as compared to coculture with unmodified BW5147 cells (FIG. 1B, #5) after 30 min of coculturing. Following 1 hour of coculturing, both BWαTrkB-GFP-Mut (FIG. 1B, #9) and BWαTrkB-Myc-Mut (FIG. 1B, #10) cells induced pTrkB in RAW-TrkB cells. The level of total TrkB receptor expression and the expression level of the actin loading control were used as references.

### Example 2. Production of IL-2 by BW Cells Expressing the Chimeric Receptor Upon Binding to the TrkB Receptor of Target Cells

Next, it was investigated whether BW cells expressing the chimeric receptor with a wild type or mutant ITAM regions, can be induced to secrete IL-2. Raw264.7 cells expressing TrkB receptor and WT Raw264.7 cells were seeded on 96 well plates in DMEM based media (10% FBS, PSN). After reaching 85-95% confluency, cells were twice washed and 100 μl of DMEM media with 0.2% FBS was added for 4 hours. After 4 hours, BW5147 cells were added to the wells in 100 μl of DMEM media without FBS for overnight incubation. Supernatants from co-cultured cells were collected after 16 hours and analyzed for IL-2 with a sandwich ELISA according to the manufacturer's instructions. Samples were analyzed in triplicates.

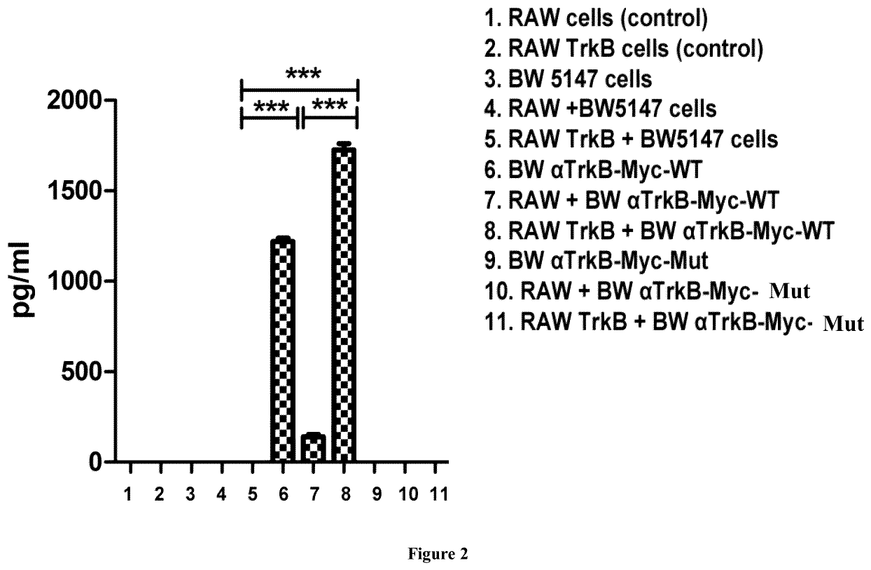

Only cells expressing BWαTrkB-Myc with wild type ITAM regions were able to produce IL-2 μpon binding with RAW cells overexpressing TrkB receptor (FIG. 2, #8). Incubation with WT RAW cells, which endogenously express low amount of TrkB receptor, induced significantly lower level of IL-2 (FIG. 2, #7). No activation of BW cells was observed when the receptor contained mutated zeta chain, regardless of whether the incubation was with WT RAW cells (FIG. 2, #10) or RAW cells overexpressing TrkB receptor (FIG. 2, #11).

### Example 3. Expression of Chimeric Receptor After Transfection into T Cells

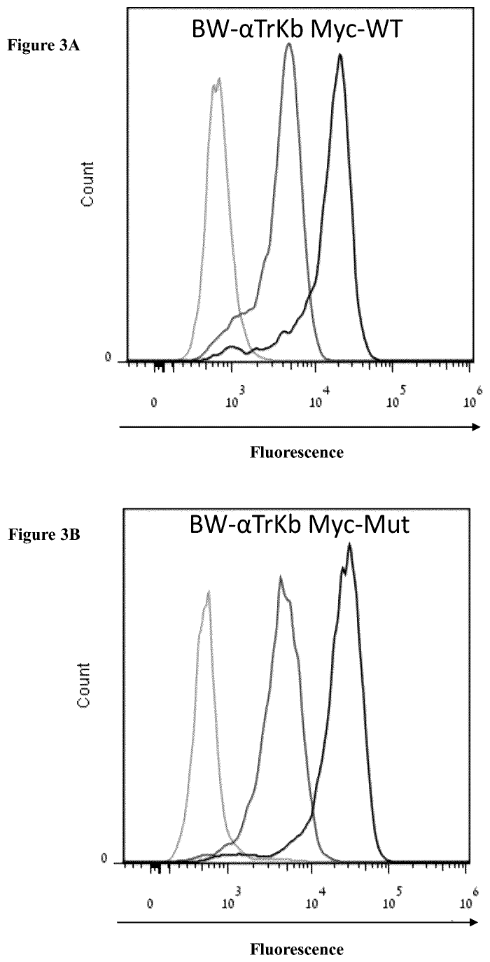

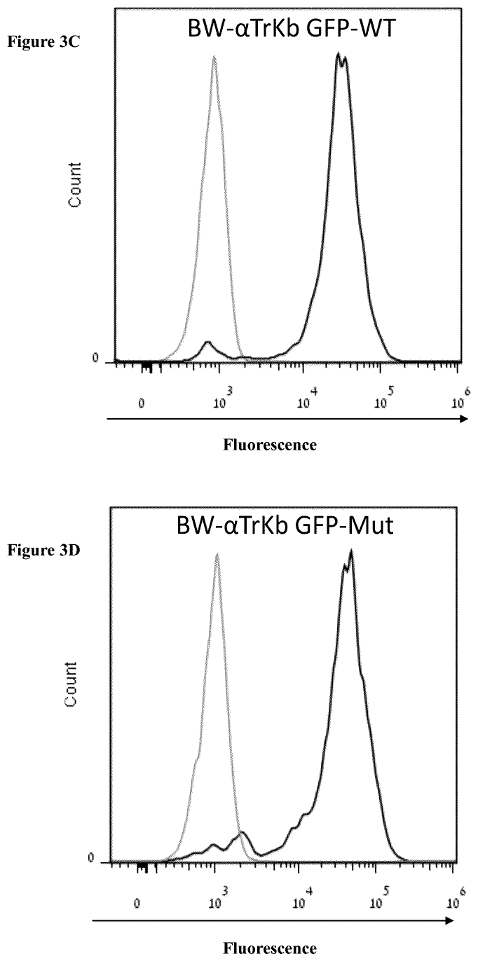

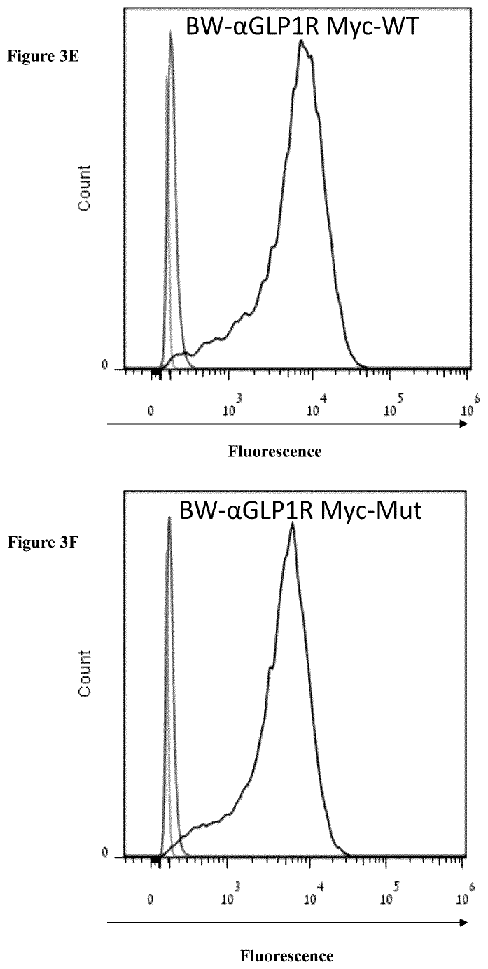

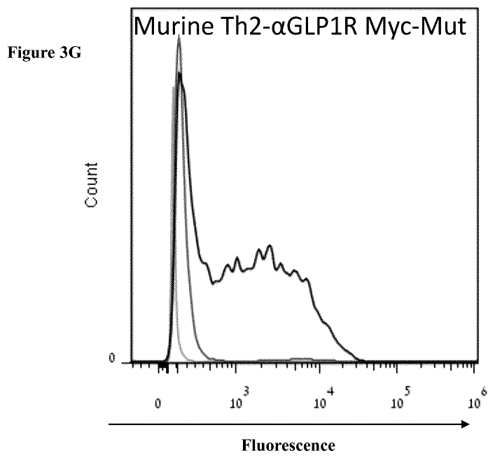

In order to assess the membrane-associated expression of the chimeric receptors on T cells the murine T cell line BW5147.3 (ATCC® TIB-47) or primary murine T-helper cells were used. Cells were transduced with recombinant retroviral vectors encoding six different chimeric receptors. 48-72 hours following transduction, cells were sorted for receptor-positive-cells using either staining for Myc expression (when Myc Tag was part of the chimeric receptor) or for GFP expression (when GFP was part of the chimeric receptor) (FIG. 3A-G). Distinct high surface expression of the chimeric receptor was observed in all cases. Specifically, αTrkB-Myc-WT (FIG. 3A, SEQ ID NO: 17), αTrkB-Myc-Mut (FIG. 3B, SEQ ID NO: 18), αGLP1R-Myc-WT (FIG. 3E, SEQ ID NO: 21), αGLP1R-Myc-Mut (FIG. 3F, SEQ ID NO: 22), αTrkB-GFP-WT (FIG. 3C, SEQ ID NO: 19), and αTrkB-GFP-Mut (FIG. 3D, SEQ ID NO: 20), were highly expressed on the surface of BW cells. Further, αGLP1R-Myc-Mut (FIG. 3G, SEQ ID NO: 22) was also shown to be highly expressed on the surface of transduced murine T-helper cells.

### Example 4. Activation of BW Cells Expressing the Chimeric Receptor with Plastic-Bound Anti-Myc Antibody

In order to test whether plastic-bound anti-Myc antibody can activate T cells expressing the Myc containing chimeric proteins to secrete IL-2 the following experiment was performed. Six different Myc-containing constructs were expressed in BW cells: the four Myc containing constructs referenced in Example 3, as well as aGH-Myc-WT (SEQ ID NO: 23), aGH-Myc-Mut (SEQ ID NO: 24). The six resultant cell lines were incubated for 16-18 hours with titrated amounts (2500 to 4.8 ng/ml in the coating solution) of plastic-bound commercial anti-Myc Ab (50000 cells/96 plate well). Anti-CD16 (2500 ng/ml in coating sol.) was employed as negative control to anti-Myc. Following overnight incubation, mIL-2 levels in the supernatant were determined by commercial ELISA kit.

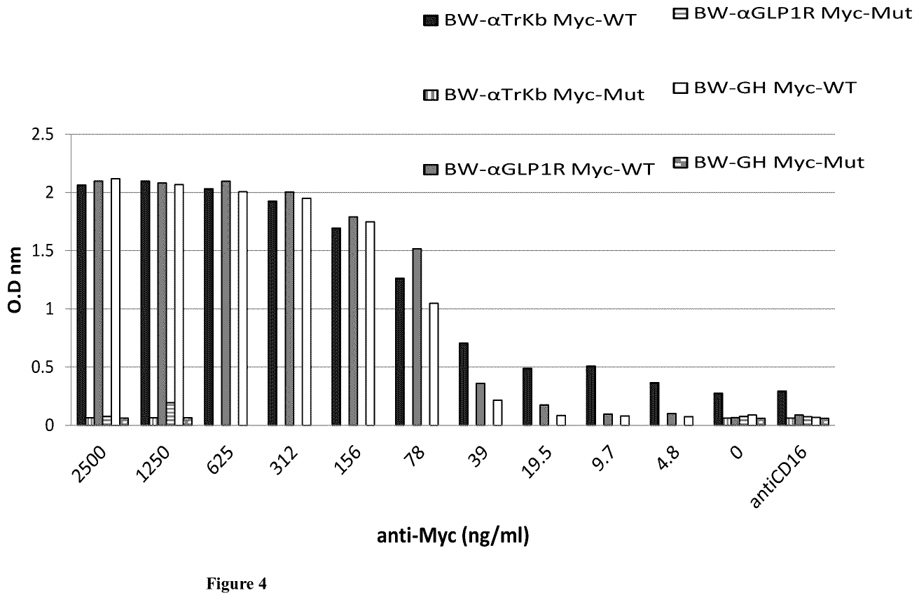

Clear activation (based on IL-2 secretion) was observed in all cells expressing constructs containing the WT zeta chain and further the intensity of Il-2 secretion was directly correlated with the amount of plastic-bound anti-Myc antibody employed (FIG. 4). No activation was observed when the expressed receptor contained a mutated zeta chain, even at the highest concentrations of the anti-Myc antibody used.

It was thus concluded that the chimeric receptors containing Myc-Tag and WT zeta chain combined with any one of the anti-TrKb V_{L}-linker-V_{H} sequence, the anti GLP1R V_{L}-linker-V_{H} sequence or the growth hormone (GH) sequence, are effectively activated by plastic-bound anti-Myc. This result shows that the chimeric receptor is functional and can be mobilized and aggregated on the membrane of the T cell to generate a functional synapse upon the presence of appropriate signal from the target side (mimicked here by plastic-bound anti-Myc antibody). Further, it is evident that the six tyrosine (Y) to phenylalanine (F) single amino acid mutations on the zeta chain are sufficient to prevent any activation by the T cell following interaction with the target.

### Example 5. Activation of T Cells Expressing αGLP1R Chimeric Receptor with Target Cells Carrying GLP1R

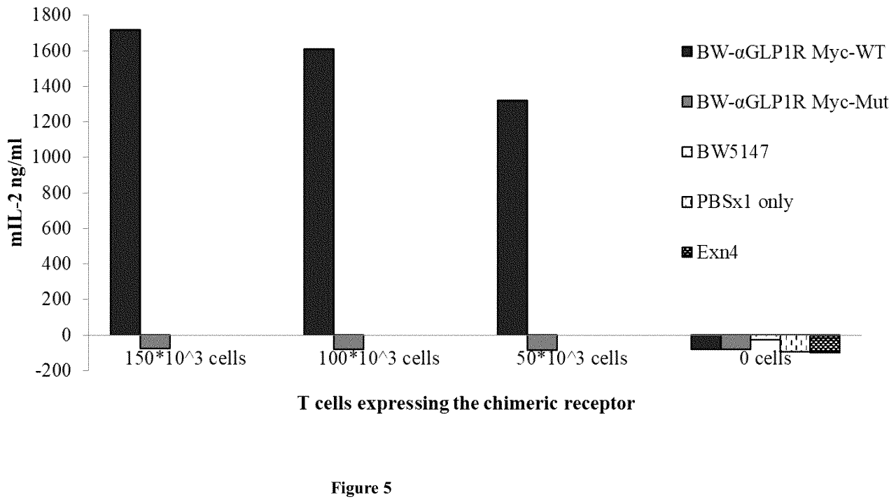

Next the ability of cells expressing GLP1R (CHO-GLP1R, Cat. No. MOO451, GenScript, Piscataway, NJ, USA), to activate T cells expressing the chimeric anti-GLP1R receptor to secrete IL-2 was examined. Shown in FIG. 5 are T cells expressing αGLP1R-Myc-WT or αGLP1R-Myc-Mut, T cells with no chimeric receptor (BW5147), and CHO-GLP1R with no T cells added (PBS). The GLP1R agonist Exendin 4 (Exn 4) was also added in place of T-cells as a control. First, commercial GLP1R-expressing CHO cells were pre-seeded and allowed to attach for 24 hours. Following incubation with T-cells or controls for 18 hours, supernatant was harvested, and murine IL-2 levels were determined by commercial ELISA kit.

Clear activation is observed (based on IL-2 secretion) only when the T cells were expressing αGLP1R-Myc-WT and this activation (IL-2 secretion) was directly correlated to the amount of T cells added (FIG. 4). No activation was measured for all other cases including the case in which T cells expressing the chimeric receptor αGLP1R-Myc-mut were added. These results corroborate the assertion that mutated zeta chain does not allow for activation of the T-cell regardless of the manner of engagement of the chimeric protein.

### Example 6. Expression of mTNFα from Target RAW Cells Incubated with Effector Cells Expressing the Appropriate Chimeric Receptor

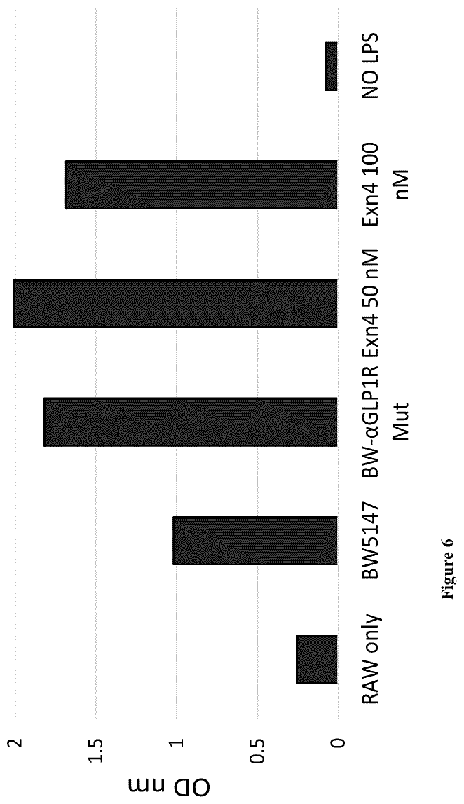

Knowing that BW cells expressing αGLP1R-Myc-Mut cannot be themselves activated, it was investigated whether these cells can activate cells expressing GLP1R to secret mTNFα. RAW264.7 cells (RAW 264.7 ATCC® TIB-71TM) are a murine macrophage/monocyte cell line that naturally expresses the murine GLP1R. Activation of GLP1R is known to induce TNFα secretion by these RAW cells. Following co-incubation with WT BW cells, BW cells expressing the chimeric anti-GLP1R receptor, or the GLP1R agonist Exn4 supernatant from the Raw cells was harvested and murine TNFα levels in the supernatant were determined by commercial ELISA kit (FIG. 6).

Clear activation of target RAW cells was observed (based on mTNFα) only when T cells that are expressing the αGLP1R-Myc-Mut were co-incubated with the target RAW cells. Low activation level and no activation of RAW cells were observed when T cells were not added (Raw only (treated with low LPS concentration—1 ug/ml) and RAW with no LPS treatment (no-LPS) columns, respectively) Some activation was observed when T cells that do not express the receptor are added (BW5147), but it is significantly less as compared to addition of T cell expressing the anti-GLP1R receptor. A positive control in this experiment that proves the effectiveness of the setup, is the addition of GLP1R agonist Exendin4 (Exn4) which induces TNFα expression that is on par with the chimeric receptor.

Thus, it is concluded that T cells expressing chimeric anti-GLP1R receptor containing the mutant zeta chain are effective at activating target cells expressing the GLP1R receptor through the GLP1R receptor. This result proves that the chimeric receptor is functional, even with the zeta mutated, and is capable of inducing potent responses from target cells expressing the appropriate receptor.

### Example 7. Blocking Negative Regulation of Immune Cells Using Anti PD-1 SCAAB

PD1-positive NK cells are incubated with immune cells expressing an anti-PD1 SCAAB (SEQ ID NO: 28). Next, PD-1L positive tumor cells are added and NK activation is measured. Alternatively, an antigen-mediated T-cell activation assay is used to assay NK cell activation.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A pharmaceutical composition that does not kill cells not of the composition, consisting essentially of a modified cell wherein said cell is an immune cell selected from a CD4+ T-cell, a B-cell, an antigen presenting cell and a dendritic cell, and wherein said cell comprises a chimeric antigen-recognizing receptor (CAR) wherein an intracellular domain of said CAR does not comprise an immunoreceptor tyrosine-based activation motif (ITAM) domain and said modified cell does not kill cells bound by said CAR; and a pharmaceutically acceptable carrier, excipient, or adjuvant.

2. The pharmaceutical composition of claim 1, wherein said composition comprises at least 1 million modified cells.

3. The pharmaceutical composition of claim 1, wherein said intracellular domain of said CAR comprises an intracellular domain of any protein other than CD3, CD28, OX-40 CD80, CD86 and a T-cell receptor (TCR).

4. The pharmaceutical composition of claim 1, wherein said intracellular domain of said CAR comprises an artificial amino acid sequence of at least 2 and at most 500 amino acids comprising at least one positively charged amino acid within 5 amino acids from an end of a transmembrane domain of said CAR.

5. A method of modulating signaling by a target receptor in a target cell in a population of cells in which cells of said population of cells are not killed, the method comprising contacting said population of cells with a composition comprising a modified cell comprising a CAR wherein an intracellular domain of said CAR does not comprise an immunoreceptor tyrosine-based activation motif (ITAM) domain, and wherein the modified cell is an immune cell selected from a CD4+ T-cell, a B-cell, an antigen presenting cell and a dendritic cell and serves as a ligand and thereby modulates signaling by a target receptor in a target cell in a population without killing cells of said population bound by said CAR.

6. The method of claim 5, wherein said signaling by a target cell comprises a signaling cascade in said target cell.

7. The method of claim 5, wherein said modulating comprises inducing or inhibiting, and wherein at least one of:
a. said inducing signaling comprises phosphorylation of a residue within a signaling domain of said target receptor;
b. said inducing signaling comprises upregulation of a level of a downstream target of said target receptor; and
c. said inhibiting signaling comprises down-regulation of a level of a downstream target of said target receptor.

8. The method of claim 5, wherein said intracellular domain of said CAR comprises an intracellular domain of any protein other than CD3, CD28, OX-40 CD80, CD86 and a T-cell receptor (TCR).

9. The method of claim 5, wherein said intracellular domain of said CAR comprises an artificial amino acid sequence of at least 2 and at most 500 amino acids comprising at least one positively charged amino acid within 5 amino acids from an end of a transmembrane domain of said CAR.

10. The method of claim 5, wherein said target receptor is at least one of:
a. associated with a disease or disorder; and
b. selected from GHR, GLP1R, TrkB, and PD-1.

11. The method of claim 5, wherein said modified cell is derived from a primary human cell from a human donor, and wherein said modified primary human cell is suitable for use in human therapy.

12. The method of claim 5, wherein said modulating treats a disease or disorder in a subject, said contacting comprises administering said cell expressing said CAR to said subject, and at least one of:
a. said disease or disorder is selected from Alzheimer's disease, depression, and memory loss, said target receptor is TrkB and said CAR is an agonist of TrkB;
b. said disease or disorder is selected from amyotrophic lateral sclerosis (ALS), epilepsy, brain cancer, said target receptor is TrkB and said CAR is an antagonist of TrkB;
c. said disease or disorder is selected from, diabetes, obesity, glycogen storage disease, Parkinson's disease, mitochondrial myopathy, stroke, myocardial infarction, cardiac ischemia, and coronary artery disease, said target receptor is GLP1R and said CAR is an agonist of GLP1R;
d. said disease or disorder is selected from growth hormone deficiency, Turner syndrome, and Prader-Willi syndrome, said target receptor is GHR and said CAR is an agonist of GHR;
e. said disease or disorder is selected from acromegaly and GHR positive cancer, said target receptor is GHR and said CAR is an antagonist of GHR;
f. said disease or disorder is selected from: rheumatoid arthritis, psoriasis, Graves' disease, immune-mediated inflammation, and celiac disease, said target receptor is PD-1 and said CAR is an agonist of PD-1; and
g. said disease or disorder is selected from lupus and PD-L1 positive cancer, said target receptor is PD-1 and said CAR is an antagonist of PD-1.

13. The method of claim 12, wherein said provided cell is at least one of:
a. autologous to said subject;
b. allogenic to said subject;
c. derived from a primary human cell from a human donor, and wherein said modified primary human cell is suitable for use in human therapy; and
d. a primary cell extracted from said subject.

14. The method of claim 5, wherein said composition consists of said modified cell and a pharmaceutically acceptable carrier, excipient, or adjuvant.

15. A method of producing an agent that modulates signaling by a target receptor in a target cell without killing said target cell, the method comprising:
a. providing a cell determined to not kill said target cell upon binding said target cell;
b. selecting at least one protein domain determined to bind to said target receptor;
c. generating a CAR comprising said selected at least one protein domain or a nucleic acid molecule encoding said CAR, wherein an intracellular domain of said CAR does not comprise an immunoreceptor tyrosine-based activation motif (ITAM) domain;
d. expressing in a plasma membrane of said cell said generated CAR to produce a modified cell;
e. confirming binding of said modified cell to said target receptor on said target cell without killing said target cell upon binding; and
f. confirming modulation of signaling by said target receptor in said target cell;
thereby generating an agent that modulates signaling by a target receptor in a target cell without killing said target cell.

16. The method of claim 15, wherein step (b) comprises selecting at least one protein domain that upon binding modulates said target receptor and wherein said method further comprises confirming said agent modulates signaling by said target receptor.

17. The method of claim 15, wherein said cell determined not to kill said target cell upon binding said target cell is selected from a CD4+ T-cell, a B-cell, a myeloid cell, a macrophage, a monocyte, a neutrophil, an antigen presenting cell and a dendritic cell.

18. The method of claim 15, wherein said confirming binding without killing comprises measuring secretion of interleukin 2 (IL-2) or tumor necrosis factor alpha (TNFα) from said modified cell and confirming binding to said target receptor on said target cell does not increase secretion.

19. The method of claim 15, wherein said intracellular domain of said CAR comprises an intracellular domain of any protein other than CD3, CD28, OX-40 CD80, CD86 and a T-cell receptor (TCR).

20. The method of claim 15, wherein said intracellular domain of said CAR comprises an artificial amino acid sequence of at least 2 and at most 500 amino acids comprising at least one positively charged amino acid within 5 amino acids from an end of a transmembrane domain of said CAR.
